SEQUENCE LISTING (6 sequences)

SEQ ID NO: 1 - protein, 15 aa, hamster
IGLHDPSHGTLPNGS

SEQ ID NO: 2 - protein, 19 aa, Hamster
IGLHDPSHGTLPNGSGWKW

SEQ ID NO: 3 - protein, 19 aa, Hamster
QYIWIGLHDPSHGTLPNGS

SEQ ID NO: 4 - protein, 19 aa, Hamster
IWIGLHDPSHGTLPNGSGW

SEQ ID NO: 5 - protein, 17 aa, Hamster
CIGLHDPSHGTLPNGSC

SEQ ID NO: 6 - protein, 21 aa, Hamster
CIWIGLHDPSHGTLPNGSGWC

(19) United States
(12) Patent Application Publication
(10) Pub. No.: US 2016/0002310 A1
(43) Pub. Date: Jan. 7, 2016

(54) MODIFIED INGAP PEPTIDES FOR TREATING DIABETES

(71) Applicant: THE ROYAL INSTITUTE FOR THE ADVANCEMENT OF LEARNING/MCGILL UNIVERSITY, Montreal (CA)

(72) Inventor: Lawrence ROSENBERG, Montreal (CA)

(21) Appl. No.: 14/768,452

(22) PCT Filed: Feb. 14, 2014
(86) PCT No.: PCT/CA2014/050104

§ 371 Date: Aug. 17, 2015

Related U.S. Application Data
(60) Provisional application No. 61/765,203, filed on Feb. 15, 2013.

(51) Int. Cl. C07K 14/47; A61K 38/10; A61K 38/17; C07K 7/08; A61K 9/00

(52) U.S. Cl. CPC C07K 14/4733; C07K 7/08; A61K 9/0019; A61K 38/1793; A61K 38/10; A61K 38/1709; A61K 38/00

(57) ABSTRACT

Novel INGAP peptides for prevention or treatment of diabetes are provided herein, as well as compositions and methods of use thereof. In particular, a 19 amino acid peptide of INGAP which possesses β-cell neogenesis and insulin potentiating activities and is sufficiently stable for in vivo use is described.

### PRIORITY CLAIM

This application claims priority to PCT application PCT/CA2014/050104 filed on Feb. 14, 2104 which claims priority to U.S. provisional patent application No. 61/765,203, filed on Feb. 15, 2013,the contents of which are expressly incorporated by reference.

### FIELD OF THE INVENTION

This invention relates to INGAP peptides with islet β-cell neogenic or regenerative activity, and compositions and methods thereof for the treatment and prevention of diabetes.

### BACKGROUND

Diabetes mellitus affects over 100 million individuals worldwide. In the U.S., the estimated healthcare costs of those affected by diabetes is approximately 136 billion dollars annually. Diabetes mellitus is a disorder of the metabolism that is characterized by the inability of the pancreas to secrete sufficient amounts of insulin, which results in large fluctuations in blood glucose levels and can have both short- and long-term physiological consequences. Long-term complications arising from elevated blood glucose levels (hyperglycemia) in patients with Type 1 diabetes (insulin-dependent diabetes mellitus, or IDDM) include retinopathy, neuropathy, nephropathy and other vascular complications. Low glucose levels (hypoglycemia) can lead to diabetic coma, seizures, accidents, anoxia, brain damage, decreased cognitive function, and death.

Type 2 diabetes, also known as non-insulin dependent diabetes mellitus or NIDDM, is a progressive disease characterized by impaired glucose metabolism resulting in elevated blood glucose levels. Patients with Type 2 diabetes exhibit impaired pancreatic beta-cell function resulting in failure of the pancreatic beta-cells to secrete an appropriate amount of insulin in response to a hyperglycemic signal, and resistance to the action of insulin at its target tissues (insulin resistance).

Current treatments of Type 2 diabetes aim to reverse insulin resistance, control intestinal glucose absorption, normalize hepatic glucose production, and improve beta-cell glucose sensing and insulin secretion. Because of the shortcomings of current treatments for diabetes, new treatments for Type 1 and Type 2 diabetes, as well as new diagnostic and prognostic methods, are highly desirable.

Increasing evidence indicates that inadequate β-cell mass underlies Type 1 and Type 2 diabetes. Therefore, regeneration of β-cells in diabetic patients is an important goal of diabetes research. In recent years, there has been increasing interest in the development of new strategies to induce β-cell regeneration and new islet formation in situ (Baggio, L. L. and Drucker, D. J., 2006, Annu Rev Med, 57:265-281). Identification of bioactive molecules with the capacity to stimulate expansion of the remaining β-cell mass or with islet neogenic activity is therefore crucial for harnessing the regenerative potential of the native pancreas.

Islet Neogenesis Associated Protein (INGAP) is a 16.8 kDa protein originally identified in a crude extract from a partially obstructed hamster pancreas (Rosenberg, L., et al., 1988, Diabetes, 37: 334-341; U.S. Pat. No. 5,834,590). INGAP is expressed in the pancreas and duodenum and has been shown to induce islet neogenesis in several species (Rosenberg, L., et al., 1996, Diabetologia, 39: 256-262; Rosenberg, L., et al., 2004, Ann Surg 240: 875-884). Structurally, INGAP is a member of the Reg family of secreted C-type lectins that comprises more than 25 members, classified into 4 subfamilies based on the primary sequence (Zhang, Y. W., et al., 2003, World J Gastroenterol, 9: 2635-2641; Okamoto, H., 1999, J Hepatobiliary Pancreat Surg 6: 254-262).

INGAP belongs to the large Reg 3 subfamily that has been identified in predominantly gastrointestinal tissues (pancreas, stomach, liver) in rat, mouse, hamster and humans (Rafaeloff, R., et al., 1997, J Clin Invest 99: 2100-2109). Despite the ubiquity of Reg proteins, not much is known about their functions or mechanisms of action. While Reg 1 is believed to be a β-cell mitogen, much less is known about the functions of the Reg 3 family.

A number of studies suggest that Regs may bind specific cell-surface receptors and activate multiple signaling pathways. One argument in favor of this receptor hypothesis is that biological activity of INGAP appears to be mediated by a 15 amino acid fragment of the protein (amino acids 104-118), namely INGAP peptide (INGAP-P), which consists of a highly conserved IGLHDP motif and a unique sequence SHGTLPNGS not found in other members of the Reg family (Rafaeloff, R., et al., 1997, J Clin Invest 99: 2100-2109). Synthetic INGAP peptide has been demonstrated to be as effective as the protein in inducing new islet formation and reversing streptozotocin-induced diabetes in hamsters and mice (Rosenberg, L., et al., 1996, Diabetologia, 39: 256-262; Rosenberg, L., et al., 2004, Ann Surg 240: 875-884) and is, therefore, a possible ligand for the receptor. Biological effects of a synthetic INGAP-P have been extensively studied both in vitro and in vivo. To date, it has been shown that INGAP-P: 1) induces in vitro regeneration of functional human islets from dedifferentiated, islet-derived duct-like structures (Jamal, A. M., et al., 2005,Cell Death Differ, 12: 702-712); 2) dose dependently stimulates expansion of β-cell mass in rodents, dogs and cynomolgus monkeys (Lipsett, M., et al., 2007,Cell Biochem Biophys, 48: 127-137; Pittenger, G. L., et al., 2007, Pancreas, 34: 103-111); and 3) increases insulin secretion and β-cell size and upregulates expression of several genes related to β-cell function in rat neonatal islets in vitro (Barbosa, H., et al., 2006, Regul Pept, 136: 78-84; Borelli, M. I., et al., 2005, Regul Pept, 131: 97-102). These important results were followed by clinical trials to investigate its efficacy and safety in humans, in which INGAP-P was found to have a signal effect with an improvement of glucose homeostasis confirmed by glycosylated hemoglobin (HbA1C, or A1C) reduction at 90 days in patients with Type 2 diabetes and by a significant increase in C-peptide secretion in patients with Type 1 diabetes (Dungan, K. M., et al., 2009, Diabetes Metab Res Rev, 25: 558-565).

Despite these data which suggest that INGAP-P possesses both islet-neogenic and insulinotropic activities, it is apparent from animal studies and human trials that the 15-mer of INGAP (INGAP-P) lacks stability. Accordingly, it must be administered in a very large dose to reach a required serum and tissue threshold level. Poor stability also leads to problems with, for example, drug formulation, patient acceptance of local injection site reactions, and high cost. There is a need therefore for an INGAP analogue with comparable or greater bioactivity and/or greater stability or a longer half-life in vivo, compared to INGAP-P.

### SUMMARY OF THE INVENTION

Previously we identified a pancreatic protein called Islet Neogenesis Associated Protein (INGAP), which is a member of the cross species mammalian family of REG3 proteins (see FIG. 19), and can induce ductal cells to differentiate into β-islet cells in a hamster model of islet regeneration (Rosenberg, L. et al., J Surg Res, 1983, 35: 63-72; Rosenberg, L. et al., Diabetes, 1988, 37: 334-341; Rosenberg, L. et al., Diabetologia, 1996, 39: 256-262). A 15-mer peptide fragment of INGAP protein containing amino acids 104-118 (INGAP-P) was identified as a bio-active center of INGAP.

Both INGAP and INGAP-P have been shown to induce ductal cells to differentiate into islets. In a human in vitro model of islet regeneration, INGAP-P induces increased expression of the pancreatic development transcription factor, PDX-1 and concurrent formation of new islets (Jamal, A. M., et al., Cell Death Differ., 2003, 10: 987-996; Jamal, A. M., et al., Cell Death Differ., 2005, 12: 702-712). In animal models, INGAP-P induces duct cell proliferation in vitro and islet cell regeneration from cells associated with the ductal epithelium, leading to new islet formation in the normal adult mouse, hamster, and dog pancreas. In the STZ-treated C57BL/6J mouse model of diabetes, INGAP-P reversed the diabetic state (Pittenger, G. L., et al., Pancreas, 2007, 34: 103-111; Rosenberg, L., et al., Ann. Surg., 2004, 240: 875-884 (2004); Kapur, R. et al., INGAP Induces Duct Cell Proliferation In Vitro and beta Cell Formation in Normal Non Diabetic Mice, 71st ADA Meeting, San Diego, 2011). In the NOD mouse model of established (not new-onset) autoimmune T1DM, INGAP-P in combination with the immune modulator IL-12 inhibitor, lisofylline, induced remission of hyperglycemia and elimination of the need for insulin pellets (Tersey, S. A. et al., Unique Drug Combination for Reversal of Type 1 Diabetes, 68th Scientific Sessions American Diabetes Association (ed. ADA), San Fransisco, Calif., 2008; Tersey, S. A. et al., Journal of Diabetes Mellitus, 2012, in press). Histologic examination confirmed evidence of new islets.

In human trials, INGAP-P has been evaluated in Phase 1 and 2 studies of both T1DM and T2DM patients (Dungan, K. M. et al., Diabetes Metab Res Rev, 2009, 25: 558-565). Once-daily injections of INGAP-P for 3 months caused a statistically significant increase in stimulated C-peptide secretion in T1DM patients, and a trend towards increased C-peptide levels in T2DM patients. Glycosylated hemoglobin (HbA_{1c}) decreased by −0.6% (p<0.0125) in T2DM patients and by −0.4% (p<0.06) in T1DM patients.

Despite these highly promising results, INGAP-P's relatively short plasma half-life continues to present challenges for use of INGAP-P as a therapeutic.

Accordingly, there are provided herein INGAP peptides which retain one or more biological activities of INGAP-P and are suitable for development as a therapeutic. In an embodiment, peptides of the invention have comparable or greater bioactivity and/or greater stability or a longer half-life in vivo, compared to INGAP-P.

In an embodiment, there is provided herein a peptide comprising the sequence set forth in SEQ ID NO:4 or SEQ ID NO:6.

In another embodiment, there is provided herein a peptide consisting of the sequence set forth in SEQ ID NO:4 or SEQ ID NO:6.

In some embodiments, a peptide of the invention induces pancreatic β-cell neogenesis, induces pancreatic β-cell regeneration, improves glucose homeostasis and/or reverses hyperglycemia in a subject.

Analogs, homologs, fragments or variants of peptides of the invention are also provided herein, wherein the analog, homolog, fragment or variant has a biological activity of the peptide. Analogs, homologs, fragments or variants may have at least 80%, at least 85%, at least 90%, at least 95% at least 98%, or at least 99% sequence identity to the peptide of the invention. The biological activity may be, for example, cell or receptor binding specificity of the peptide, ability to induce pancreatic β-cell neogenesis, ability to induce islet cell regeneration, ability to improve glucose homeostasis and/or ability to reverse hyperglycemia in a subject.

Nucleic acid molecules comprising a nucleic acid sequence encoding peptides of the invention or analogs, homologs, fragments or variants thereof are also provided. A nuceic acid molecule may be operably linked to an expression control sequence to form an expression vector, wherein said expression vector is propagated in a suitable cell.

Pharmaceutical compositions comprising peptides or analogs, homologs, fragments or variants thereof of the invention, and a pharmaceutically acceptable carrier or excipient, are also provided. In an embodiment, compositions are adapted for administration orally. In another embodiment, compositions are adapted for administration by injection.

In another embodiment, there is provided a method for preventing or treating a pancreatic condition or disease comprising administering a peptide or analog, homolog, fragment or variant thereof or a composition of the invention to a subject in need thereof. In an embodiment, the condition or disease is a metabolic disorder. In another embodiment, the condition or disease is a β-cell associated disorder. In a further embodiment, the condition or disease is Type 1 diabetes, Type 2 diabetes or a complication of diabetes.

In some embodiments, β-cell death by apoptosis or necrosis is prevented or inhibited in a subject by administering peptides or analogs, homologs, fragments or variants thereof, or compositions, of the invention. In other embodiments, functionality of pancreatic cells is improved or restored in a subject, plasma insulin levels are increased in a subject, number or size of pancreatic β-cells is increased in a subject, β-cell regeneration from pancreatic ductal cells is stimulated in a subject, glucose homeostasis is restored or improved in a subject, and/or hyperglycemia is reversed in a subject.

Peptides and compositions of the invention may be administered by injection, orally, intravenously, intraperitoneally, intramuscularly or subcutaneously. In an embodiment, peptides and compositions are administered orally, once-a day.

In a particular embodiment, a subject is a human.

In some embodiments, peptides of the invention are administered with a second therapeutic agent. A second therapeutic agent may be administered concomitantly with a peptide of the invention, or a second therapeutic agent and a peptide may be administered sequentially. In an embodiment, a second therapeutic agent is a therapeutic for Type 1 or Type 2 diabetes. In another embodiment, a second therapeutic agent is anakinra

Pharmaceutical compositions for treatment of pancreatic insufficiency, comprising a peptide of the invention and a pharmaceutically acceptable carrier or excipient, are also provided. In an embodiment, a peptide or composition is capable of stimulating β-cell regeneration from pancreatic ductal cells. In another embodiment, a peptide or composition has a biological activity of mammalian INGAP protein. In one embodiment, a biological activity is ability to stimulate pancreatic duct-like cells or duct-associated cells to grow and proliferate.

Nucleic acid molecules encoding peptides of the invention or analogs, homologs, fragments or variants thereof described herein are also provided. Nucleic acid molecules may, for example, be linked to an expression control sequence to form an expression vector, wherein said expression vector is propagated in a suitable cell.

In yet another embodiment, the present invention provides pharmaceutical compositions comprising peptides or analogs, homologs, fragments or variants thereof described herein and a pharmaceutically acceptable carrier or excipient.

There are also provided herein methods for preventing or treating a pancreatic condition or disease comprising administering a peptide or analog, homolog, fragment or variant thereof of the invention to a subject in need thereof. In methods provided herein, a subject may be for example a rodent, canine, pig, primate or human.

In an embodiment, a condition or disease is a metabolic disorder, for example a β-cell associated disorder. A condition or disease may be Type 1 diabetes, Type 2 diabetes or a complication of diabetes.

In other embodiments, β-cell apoptosis is prevented or inhibited in a subject; functionality of pancreatic cells is improved or restored in a subject; plasma insulin levels are increased in a subject; and/or number or size of pancreatic cells is increased in a subject. In a particular embodiment, the pancreatic cells are β-cells.

In yet another embodiment, β-cell neogenesis is stimulated and/or glucose homeostasis is improved in a subject and/or insulin is potentiated in a subject.

### BRIEF DESCRIPTION OF THE DRAWINGS

Particular embodiments of the present invention will now be explained by way of example and with reference to the accompanying drawings.

FIG. 1 shows effect of INGAP on proliferation in RIN-m5F cells. (A): INGAP increased BrdU incorporation. RIN-m5F cells were treated with indicated amounts of INGAP-P or r-INGAP for 24 h in chamber slides. Exendin 4 (Ex4) and EGF were used as controls. 50 μM BrdU was added for the last three hours of treatment followed by fixation in methanol and immunostaining for BrdU. Data are presented as a ratio of BrdU(+) cells (%) in INGAP-treated to untreated control. Results are means±S.E. of three independent experiments (*: p<0.05, §: p<0.001, compared to untreated control). (B): INGAP induced phosphorylation of Erk½ in RIN-m5F. RIN-m5F cells (1×10⁶) plated in 60 mm tissue culture plates were treated with INGAP for the times indicated. Blots (30 μg protein) were probed with anti-Erk½-Phospho (Thr202/Tyr204) antibody, followed by stripping/reprobing with anti-total Erk½ antibody (Cell Signaling) and quantified by densitometry, using ImageJ software. (C): Quantification of relative Erk½ phosphorylation measured as a ratio of Phospho-Erk½ to total Erk½ and shown as a Fold Change relative to the 0 min time point. Results are means±S.E. of three to eight independent experiments (*: p<0.05, §: p<0.01, ¶: p<0.001; †: for the indicated time points only, two experiments were conducted).

FIG. 2 shows fluorescently labeled rINGAP forms caps on the cell surface. RIN-m5F cells plated in chamber slides were incubated for the times indicated, at 37° C. or on ice, with 50nM rINGAP labeled with DyLight 488 reactive dye and fixed in 4% paraformaldehyde on ice. (A): Cells were prechilled on ice for 15 min and incubated with rINGAP and CTB (AlexaFluor-594, 5 μg/ml, Invitrogen) for 30 min. (B): same with Transferrin (25 m/ml, Texas Red, Invitrogen). (C), (D): 1 h incubation with CTB and Transferrin, respectively, at 37° C. (E), (F): Cells were incubated for 5 h or 24 h with labeled rINGAP and co-stained with 50 nM LysoTracker Red DND99 (LT, Invitrogen) for the last hour. Nuclei were counterstained with DAPI included in the mounting medium (Prolong Gold, Invitrogen). Images were taken with an Olympus FV10i confocal microscope. Bars are 20 μm.

FIG. 3 shows binding and internalization of fluorescently labeled rINGAP was partially inhibited by 100 nM Wortmannin and Cytochalasin D, suggestive of macropinocytosis. RIN-m5F cells plated in chamber slides were incubated for 5 h with 50 nM r-INGAP labeled with DyLight 488 reactive dye in the presence of Wortmannin (100 nM) or CytochalasinD (25 m/ml) and fixed in 4% paraformaldehyde. (A): rINGAP, no inhibition; (B): negative control; (C): Wortmannin; (D): CytochalasinD. Nuclei were counterstained with DAPI included in the mounting medium (Prolong Gold, Invitrogen). Images were taken with an Olympus FV10i confocal microscope.

FIG. 4 shows FAM-labeled INGAP-P was rapidly internalized into the cytoplasm of RIN-m5F cells. Cells grown in chamber slides were treated with FAM-labeled INGAP-P for the times indicated and fixed with 4% PFA. (A), (B), (D), (F): Cells were stained with Lysotracker for 1 h, as in FIG. 2. (C), (E): Fixed cells were permeabilized with 0.1% Triton-X100 for 10 min, blocked in 5% goat serum and probed with anti-EEA1 rabbit primary antibody (Abcam, 1:200) overnight, at 4° C. followed by the secondary donkey anti-rabbit DyLight594-conjugated antibody (1:500) for 1 h at room temperature. Nuclei were counterstained with DAPI included in the mounting medium (Prolong Gold, Invitrogen). Images were taken with an Olympus FV 10i confocal microscope.

FIG. 5 shows internalization of FAM-INGAP-P is inhibited by CytochalasinD but not by Wortmannin. Cells grown in chamber slides were treated with FAM-labeled INGAP-P (16.7 μM) for 1 h in the presence of CytochalasinD (25 μg/ml) or Wortmannin (100 nM), and fixed and imaged as described herein. (A): FAM-INGAP-P; (B): DMSO control; (C): CytochalasinD; (D): Wortmannin.

FIG. 6 shows a molar excess competition assay for binding and internalization of fluorescently labeled rINGAP and INGAP-P. RIN-m5F cells plated in chamber slides were incubated with FAM-INGAP-P for 1 h (left panel) or with DyLight-488 rINGAP for 5 h (right panel) (A, B): no inhibition; (C, D): with 167 μM (10× molar excess) of INGAP-P; (E, F): with 104 (20× molar excess) rINGAP.

FIG. 7 shows involvement of Ras-Raf activation in signaling events induced by INGAP-P and r-INGAP. (A): Ras activation was measured by Ras-GTP ELISA. 1×10⁶ cells were plated in 60mm plates for 48 hours followed by a 24-h starvation in serum-free medium. Cells were treated with growth factors at 37° C., for the times indicated.

Plates were then placed on ice and washed with ice-cold PBS prior to cell lysis in 150 μl of Mg⁺ lysis buffer containing a cocktail of protease inhibitors (NEB). 10 μl of cell lysates were used for Ras-GTP ELISA and the readings were normalized by amounts of protein (DC protein assay, Biorad). Results are shown as a Fold Change relative to the 0 min time point, which is equal to 1 and is shown as a dotted horizontal line. Results are means±S.E. of at least three independent experiments (*: p<0.05, §: p<0.01, ¶: p<0.001, compared to the 0 min control). (B): Fold change in c-Raf phosphorylation, measured by Western blot/densitometry (ImageJ), as a ratio of Phospho to total cRaf and calculated relative to the 0 min time point, shown as a dotted horizontal line (=1). Results are means±S.E. of at least three independent experiments (*: p<0.05, §: p<0.01, ¶: p<0.001).

FIG. 8 shows effect of pharmacological inhibitors on Erk½ phosphorylation by INGAP, EGF and Ex-4. 1×10⁶ cells were plated in 60 mm plates for 48 hours followed by a 24-h starvation in serum-free medium. Prior to addition of growth factors, cells were pretreated for 30-40 min with the indicated inhibitors, except for Pertussis Toxin (Ptx)(24 h pretreatment). After a 10 min treatment with growth factors, cells were placed on ice and lysed, as described in the Experimental Procedures. Blots (30 μg of proteins) were incubated with Phospho-Erk½ antibody (or with total Erk antibody after stripping) and developed using ECL reagent. Quantification of Erk phosphorylation relative to total Erk and time 0 min control (=1, shown as a dotted line) was performed as described above. (A): Inhibitors of GPCR (Ptx, 100 ng/m1), adenylate cyclase (SQ22536, 250 μM) and PKA (PKi, 100nM; H89, 1μM). (B): inhibitors of PKC (Bis, 1 μM), PI3K (Wm, 100 nM), Src(PP2, 100nM), EGFR(AG1478, 100nM) and Raf Inhibitor l(R-1, 100nM), or DMSO as a solvent (*: p<0.05).

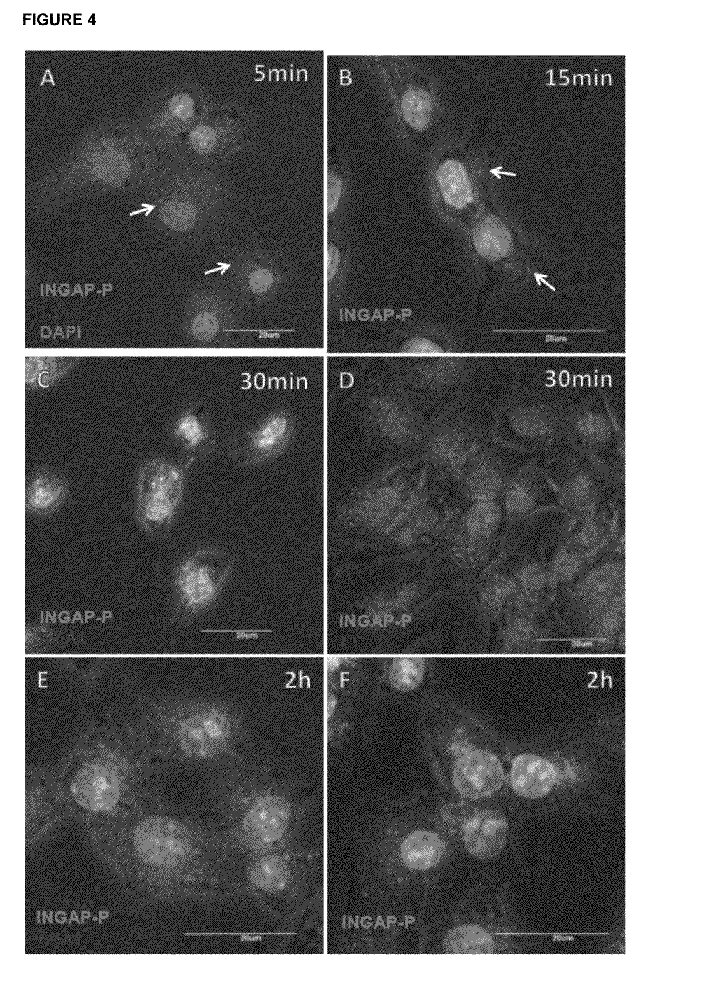

FIG. 9 shows inhibition of GPCR signaling resulted in diminished Ras activation. RIN-m5F cells grown in 60 mm plates were pretreated with Ptx for 24 h prior to addition of growth factors for 1, 3, 5 and 10 min. Cells were harvested in Mg⁺ lysis buffer and subjected to the Ras-GTP ELISA, as described in FIG. 4. Results are means±S.E. of at least three independent experiments (*: p<0.05, §: p<0.01, ¶: p<0.001).

FIG. 10 shows live imaging of rINGAP binding. A time course is shown, as follows: (A): 0 min; (B): 2 min; (C): 5 min; (D): 15 min; (E): 20 min; and (F): 30 min; white thick arrows indicate membrane-bound INGAP; thin arrows indicate intracellular INGAP; and red arrow indicates a dead cell. Images were taken with a Zeiss LSM-510 META confocal microscope.

FIG. 11 shows that rINGAP does not co-localize with either clathrin (A) or caveolin (B, C). Cells were incubated with DyLight-594-rINGAP for 1, 15 min (A,B) or 3 h (C), fixed in 4% PFA and probed with rabbit anti-clathrin or anti-caveolin antibodies overnight at 4° C., followed by detection with FITC-labeled goat anti-rabbit secondary antibody. Nuclei were counterstained with DAPI included in the mounting medium (VECTASHIELD HardSet Mounting Medium). Images were taken with a Zeiss LSM-510 META confocal microscope. Arrowheads indicate membrane-bound rINGAP, and arrows indicate intracellular rINGAP.

FIG. 12 shows internalization of DyLight 488-labeled rINGAP after 1 h of exposure fpllowed by washing and a chase period of 5h (A) or 24 h(B) without presence of labeled INGAP. LysoTracker Red DND99 (50 nM) was added 1 h prior to fixation in 4% PFA. Nuclei were counterstained with DAPI included in the mounting medium (VECTASHIELD HardSet Mounting Medium, Vector). Images were taken with an Olympus FV10i confocal microscope.

FIG. 13 shows internalization of FAM-INGAP-P after 24 h of continuous exposure (A) or 24 h chase (B) following 1 h of incubation. LysoTracker Red DND99 (50 nM) was added 1 h prior to fixation in 4% PFA. Nuclei were counterstained with DAPI included in the mounting medium (VECTASHIELD HardSet Mounting Medium Vector). Images were taken with an Olympus FV10i confocal microscope.

FIG. 14 shows INGAP-P internalization is inhibited on ice and in the presence of lipid raft inhibitor Filipin (Calbiochem). RIN-m5F cells grown in 8-well chamber slides were treated with FAM-INGAP-P for 1 h either at 37° C. (A) or on ice (B) or in the presence of 1 μg/ml Filipin. INGAP-P is shown in green. Nuclei were counterstained with DAPI (blue) included in the mounting medium (VECTASHIELD HardSet Mounting Medium). Images were taken with a Zeiss LSM-510 META confocal microscope.

FIG. 15 shows quantification of Akt phosphorylation in RIN-m5F cells treated with INGAP, EGF and Ex-4.Cell lysates from samples prepared in Mg⁺ lysis buffer for Ras-GTP ELISA were assayed by Akt ELISA (Millipore) according to the manufacturer's instructions and normalized by the amounts of protein, as described herein. Results shows a fold change relative to time 0 (=1, shown as a dotted line) and are means±S.E. of at least three independent experiments (*: p<0.05, §: p<0.01, ¶: p<0.001).

FIG. 16 shows effect of pharmacological inhibitors on proliferation of RIN-m5F cells. Cells were plated in chamber slides and subjected to 30−40 min pretreatment with the indicated inhibitors prior to the addition of growth factors and incubated for 24 h. 50 mM BrdU was added for the last three hours of treatment followed by fixation in Methanol and immunostaining for BrdU. Data are presented as a ratio of BrdU(+) cells (%) in treated versus untreated control. Results are means±S.E. of three independent experiments (*: p<0.05, †: p<0.001).

FIG. 17 shows sequence and 3D-structure of INGAP-protein. (A) shows amino acid (aa) sequence; INGAP-P is in black and underlined and the conserved flanking aa are in green; (B) shows INGAP-P is located on an external loop of rINGAP (black; adjacent IW and GW are in green); (C) shows homology between INGAP-P and corresponding peptide sequences in Reg3 proteins across species. Arrows indicate the conserved aa considered for inclusion into extended INGAP-P peptides.

FIG. 18 shows the effect of three extended INGAP-P analogues on Erk½ activation in RINm5F cells. RIN-m5F cells (1×10⁶/60 mm dish) were treated with rINGAP protein 1 nM and 10 nM, INGAP-P (15mer) or 19 mer analogues (at 1×-167 nM or 10×-1.67 μM) for 10 min. Quantification of Erk½ activation was done on Western Blots using ImageJ software and was determined as a ratio of Phospho-Erk½ (Thr202/Tyr204) to total Erk½. Data are shown as a Fold Change in treated cells relative to control (PBS) and are expressed as Mean±S.E. of 6 independent experiments for rINGAP and INGAP-P (15 mer), and 2 independent experiments for 19 mer analogues. In every experiment 2-3 separate plates were used for each treatment (*: p<0.05, **: p<0.01, ¶: p<0.001).

FIG. 19 shows binding of INGAP-19 to RINm5F cells resembles binding of rINGAP. RIN-m5F cells grown in 8-well glass chamber slides were incubated with either 50 nM DyLight488-labeled rINGAP (A), 8.35 μM INGAP-P (B) or 8.35 μM INGAP-19 (C) for 30 min, washed with PBS and fixed with 4% paraformaldehyde on ice. Slides were mounted using Prolong Gold with DAPI (Invitrogen) for counterstaining of nuclei and examined under confocal microscope Zeiss LSM 510. Arrows indicate membrane-bound rINGAP and INGAP-19, whereas arrowheads point at internalized INGAP-P. (D) shows staining with FAM alone (negative control).

FIG. 20 shows a degradation profile of INGAP-P (top) and INGAP-19 (bottom) in presence of serum. 50 μM peptides were incubated in RPMI-1640 medium with 25% FBS for the times indicated. Following ethanol precipitation of serum proteins, samples were analyzed by HPLC. To compare dynamics of peptide degradation HPLC profiles were superimposed as shown.

FIG. 21 shows time-course studies of in vitro incubation of peptides in FBS, wherein (top) shows INGAP-PC peptide and (bottom) shows INGAP-19C peptide. 50 μM INGAP-PC and INGAP 19C were incubated in RPMI-1640 medium with 25% FBS for the times indicated. Following ethanol precipitation of serum proteins, samples were analyzed by HPLC. To compare dynamics of peptide degradation, HPLC profiles were superimposed as shown. It can be seen in (B) that no degradation was seen for INGAP-19C for 48h in presence of serum.

FIG. 22 shows effect of INGAP analogue peptides on Erk½ activation in RINm5F cells. (A) shows results of a pilot study comparing analogues at the same concentration as INGAP-P (167nM; n=2); (B) shows a comparison of lower and higher doses of INGAP-P, INGAP-19 and INGAP-19C. Treatment of RINm5F cells and quantification of Erk½ activation was carried out as described for FIG. 11.

FIG. 23 shows relative effectiveness of rINGAP and INGAP 15-mer peptide (INGAP-P) in inducing islet regeneration from human islet-derived duct-like structures (DLS). Islet character (% change; the number of insulin positive structures/total number of structures after treatment) was compared to pretreatment levels (*p<0.05).

FIG. 24 shows effect of rINGAP and INGAP-P treatment on blood glucose levels in diabetic mice. C57B1/6J mice rendered chronically diabetic (glycemia approx. 27 mM) by a single ip injection of STZ (150 mg/kg) were treated for 7 weeks with rINGAP (5 m), INGAP-P (500 μg) or PBS. Data are expressed in mmol/L and are Mean ±SEM. STZ-PBS (n=5); STZ-rINGAP (n=6); and STZ-INGAP-P (n=7); *p<0.05, **p<0.01, treated vs PBS.

FIG. 25 shows INGAP induced Pdx-1 expression in human adult ductal cells. (A) shows Pdx-1 mRNA expression variation over time in HPDE cells treated with 167 nM INGAP-P, expressed as a fold-change of the time-matched untreated control. (B) shows Pdx-1 mRNA expression variations in HPDE cells treated for 15 minutes with different doses of rINGAP, expressed as a fold-change of the time-matched untreated control. (C) shows a representative Western blot of Pdx-1 expression after 24 hours in HPDE untreated cells (CTL) and cells treated with 167 nM INGAP-P. Equal amounts of total protein were loaded onto each lane (as shown with β-Actin). (D) shows a graphical representation of % increase in Pdx-1 protein, as seen in (C), quantified with ImageJ software. Data is presented as mean±SEM, *p<0.05, n=3 independent measurements.

FIG. 26 shows INGAP-P induced coordinated expression of developmental transcription factors implicated in endocrine differentiation during development. NeuroD1 (A), IA-1 (B) and MafA (C) mRNA expression variations overtime, expressed as a fold-change of time-matched untreated control (*p<0.05), are shown.

FIG. 27 shows INGAP induces expression of Insulin and Glucokinase in HPDE cells after 24 h. (A) shows insulin expression after 24 h, in absence (Ctrl) or presence (1 ×INGAP) of 167 nM INGAP-P; (B) shows glucokinase expression detected by RT-PCR after 24 h, in absence (Ctrl) or presence of 5 nM rINGAP (rINGAP) (representative gel is shown); (C) shows levels of C-peptide detected in HPDE cell lysates after 24 h in culture in absence (Ctrl) or presence of 167 nM INGAP-P (1×INGAP) or 5 nM rINGAP (rINGAP) by ELISA (*p<0.05, normalized to total protein).

FIG. 28 shows clustering HPDE cells mimic the islet-DLS-ILS model and enhance endocrine differentiation triggered by INGAP. (A) HPDE cells embedded in Matrigel formed clusters after 5 days in culture. After 10 days, the clusters became cystic. When treated for 7 days with 167 nM INGAP-P, HPDE cystic structures reverted into solid islet-like clusters (phase-contrast microscopy, representative pictures). (B) Changes in expression of Insulin, Glucagon and PPY mRNA in HPDE clusters treated for 7 days with 167 nM INGAP-P (INGAP-P) or 5 nM rINGAP (rINGAP) are shown, expressed as a fold-change of the time-matched untreated control (*p<0.05).

FIG. 29 shows matrigel-embedding of HPDE cells intensified endocrine differentiation upon INGAP treatment. Immunofluorescence analysis of HPDE clusters cultured 7 days without (CONTROL) or with 167 nM INGAP-P (INGAP) is shown: immunodetection of Cytokeratinl 9 (CK19), PDX-1,C-peptide and Glut-2 (representative pictures). Upon INGAP treatment, CK19 was abolished, PDX-1 was translocated to the nucleus (arrows), and C-peptide (arrow) and Glut 2 appeared.

FIG. 30 shows islet-to-DLS Conversion: I Morphology and Immunofluorescence. Inverted (A-C) and IF (D-F) microscopy demonstrated that islets are solid spherical structures (A) comprised mainly of insulin and β-cells (D). Through the 8-day culture period, foci of DLS formation formed and expanded (B, E), eventually replacing the islets. These DLS were hollow, cystic structures (C) that were a heterogeneous population of CK+duct epithelial cells (F). Morphological evaluation of DLS formation correlated positively with ductal (green: CK+) cell frequency (r2=0.74, p<0.001) and correlated negatively with endocrine (red: insulin/glucagon/somatostatin/PP+) cell frequency (r2=0.64, p<0.001) (G). II Apoptosis. Early DLS formation was characterized by marked apoptosis, as assessed by ELISA ((A): *p<0.05, **p<0.01 vs day 0). TUNEL-based studies of day 1 cultures indicated that apoptotic cells were predominantly β-cells (B). III DLS Proliferation. Immunohistochemical (A) and quantitative assessment (B) of BrdU incorporation indicated that DLS cells were highly proliferative relative to the relatively quiescent islet cells (*p<0.05, **p<0.01, ***p<0.001).

FIG. 31 shows Islet-to-DLS Conversion: Progenitor Marker Expression. Immunohistochemical and immunofluorescence analyses indicated that DLS cells expressed markers associated with islet progenitors, including PDX1, nestin and vimentin.

FIG. 32 shows DLS-to-ILS regeneration after INGAP-P treatment: Morphology and Immunohistochemistry. Treatment of DLS with 167 nM INGAP-P for 4 days induced the formation of ILS that expressed appropriate levels of adult islet functional markers and had downregulated CK expression relative to DLS.

FIG. 33 shows DLS-to-ILS Regeneration: Function. Assessment of insulin content (A) and glucose-induced insulin secretion (B) indicated that ILS had equivalent insulin stores and secretory capacity to the initial islets from which they were derived (*p<0.01 vs islets).

FIG. 34 shows INGAP increased HPDE cell proliferation. HPDE cells in monolayers (A) or cultured in Matrigel as clusters (B) were treated with 167 nM INGAP peptide (1×) for 7 days. Cells were then stained for the marker of proliferation, PCNA, and the percentage of PCNA+/total number of nuclei was calculated (*p≦0.05). CTRL is untreated control.

FIG. 35 shows effect of INGAP on protein kinase activation in HPDE cells using Kinetworks™ Broad Signaling Pathway Screen (KPPS 1.3, Kinexus Bioinformatics). (A-C), Kinetworks Western blot results of various phosphoprotein kinases from HPDE cells treated for 20 minutes with PBS (control), 835 nM INGAP-P, and 1 nM rINGAP, respectively; (D), statistical bar diagram of OD for various protein kinase activation after 20 minutes from control (empty bars), INGAP-P-treated (gray bars), and rINGAP-treated (black bars) cells; [symbol] , abbreviated names of protein kinases as depicted by numbers in (A-D), respectively and the corresponding fold-changes. Only significant changes are represented; a change in OD of at least 25% was considered significant.

FIG. 36 is a chart showing the effect of the 4 different analogues of INGAP on the expression of Pdx-1 by qPCR

FIG. 37 is a graph showing the effect of INGAP on cell proliferation.

FIG. 38 is a graph showing the effect of INGAP on Erk½ activation.

FIG. 39 is a graph showing the effect of INGAP on Akt activation.

FIG. 40 is a graph showing the effect of INGAP on RINm5F cells,

FIG. 41 is a graph showing the stability of INGAP peptides in serum.

FIG. 42 is a graph showing averagefasting glycemia.

FIG. 43 compares the average pancreatic weight of INGAP-treated mice was improved as compared to the control group.

FIG. 44A-H shows the histologic characteristics of the pancreas of streptozotocin-treated C57BL/6J mice with and without INGAP analogs peptide treatment.

Other features and advantages of the present invention will become apparent from the following detailed description. It should be understood, however, that the detailed description and the specific examples while indicating particular embodiments of the invention are given by way of illustration only. Various changes and modifications within the spirit and scope of the disclosure will become apparent to those skilled in the art from this detailed description.

### DETAILED DESCRIPTION OF THE INVENTION

Islet Neogenesis Associated Protein (INGAP) was discovered in the partially duct-obstructed hamster pancreas, as a factor inducing formation of new duct-associated islets. We have shown previously that a bio-active portion of INGAP, INGAP¹⁰⁴⁻¹¹⁸ peptide (also referred to herein as “INGAP-P”, “15-mer” or SEQ ID NO:1), has β-cell neogenic and insulin-potentiating activities. Recent Phase 2 clinical trials have shown that INGAP-P produced improved glucose homeostasis in both Type 1 and Type 2 diabetic patients, thus supporting the potential of INGAP as a pharmacotherapy for diabetes. However, poor stability and/or a short plasma half-life have hampered the ability to develop INGAP-P as a therapeutic.

We report herein that, to improve efficacy of INGAP-P as a medicament for treatment or prevention of diabetes and to understand its mechanism of action, we searched for clues in the full-length INGAP protein (also referred to as “rINGAP” and “r-INGAP”). We cloned rINGAP and used it to investigate signaling events induced by the protein and the INGAP-P peptide in RIN-m5 F cells. RIN-m5 F cells are a rat insulinoma cell line that responds to INGAP with an increase in proliferation. The full length recombinant protein (r-INGAP) was much more stable than the 15-mer peptide (up to 5 days in cell culture) and is 6His-tagged. The data showed that rINGAP was at least a hundred times more efficient on a molar basis than INGAP-P at stimulating proliferation of rat insulinoma RIN-m5F cells and that, although they both signal via activation of a Ras-Raf-Erk pathway, upstream signaling events may differ. We also show that binding of fluorescent-labeled rINGAP is limited to the cell surface and forms patches on the cell surface in a fashion consistent with receptor binding and clustering, whereas INGAP-P is rapidly internalized. INGAP-induced Erk½ (MAPK42/44) activation is significantly reduced by pertussis-toxin (Ptx) for both rINGAP and the 15-mer, suggesting that both rINGAP and the peptide act via a G-protein coupled receptor. Thus, the data showed that rINGAP had a much greater stability (up to 5 days in cell culture) and at least 100 times higher molar efficiency in islet regeneration, both in vitro and in vivo than INGAP-P (see FIGS. 23, 24, ).

Using X-ray crystallography, a 3 D reconstruction of rINGAP was generated. This reconstruction showed that the bioactive 15-mer peptide, INGAP-P, is part of a loop extending out from the core of the molecule (FIG. 17B). It is noteworthy that the 15-mer, INGAP-P, is a small linearized peptide. We hypothesized that the loop in the rINGAP protein may facilitate an interaction of the protein with its target cell/receptor, and that preserving the loop structure may therefore be key to bioactivity and stability of an INGAP peptide. Analysis of the protein sequence (FIG. 17A) showed that INGAP-P is flanked by highly hydrophobic amino acids sequences forming the protein core. Notably, three tryptophan residues (W) situated in immediate proximity to INGAP-P (amino acids 103,120 and 122) (FIG. 17C) and Glycine¹¹⁹ (G), are highly conserved in Reg 3 proteins across species including human.

We therefore designed and produced three extended analogues of INGAP-P that included the conserved amino acids either on one side of the peptide, or on both sides (see Table 1, FIG. 18). So as not to compromise solubility, extensions were limited to four amino acids resulting in 19-mer peptides. Extended INGAP peptides may conserve the native 3 D loop structure of INGAP. Without wishing to be bound by theory, it is believed that two hydrophobic trytophan residues at either end of a 19-mer may stabilize a loop structure of the peptide.19-mer peptides thus retain biological activity of rINGAP protein (perhaps even showing enhanced activity) and possess increased stability and/or plasma half-life as compared to INGAP-P 15-mer.

Structures of 19-mer peptides are shown in Table 1. To produce extended 19-mer INGAP peptides, 4 amino acids flanking the INGAP-P 15-mer peptide in the INGAP protein sequence were added to the INGAP-P 15-mer, as shown in Table 1 (flanking amino acids added to the 15-mer are underlined; see also FIGS. 17, 18).

TABLE 1
INGAP Peptide sequences.

| Peptide | Amino acid sequence |
|---|---|
| 15-mer | N′-IGLHDPSHGTLPNGS-C′ |
| (INGAP¹⁰⁴⁻¹¹⁸; | (SEQ ID NO: 1) |
| INGAP-P) | |
| 19-mer seq1 | N′-IGLHDPSHGTLPNGSGWKW-C′ |
| | (SEQ ID NO: 2) |
| 19-mer seq2 | N′-QYIWIGLHDPSHGTLPNGS-C′ |
| | (SEQ ID NO: 3) |
| 19-mer seq3 | N′-IWIGLHDPSHGTLPNGSGW-C′ |
| (INGAP¹⁰²⁻¹²⁰; | (SEQ ID NO: 4) |
| INGAP-19) | |
| Cyclized 15-mer | N′-CIGLHDPSHGTLPNGSC-C′ |
| (INGAP-PC) | (SEQ ID NO: 5) |
| Cyclized 19-mer | N′-CIWIGLHDPSHGTLPNGSGWC-C′ |
| (INGAP-19C) | (SEQ ID NO: 6) |

Biological activity of 19-mers in comparison to INGAP-P 15-mer and to rINGAP was investigated. As shown herein, INGAP¹⁰²⁻¹²⁰⁻ induced Erk½ (MAPK42/44) activation in cultured RIN-m5 F cells was 3 times greater than that produced by INGAP¹⁰⁴⁻¹¹⁸ and approximately double that produced by rINGAP (FIG. 18). Furthermore, we demonstrate that binding of fluorescent-labeled 19-mer (INGAP¹⁰²⁻¹²⁰) is limited to the cell surface and resembles receptor clustering in a manner similar to that visualized for rINGAP, and is distinctly different from INGAP^{104-118,} which does not appear to bind to the cell surface (FIG. 19).

Analysis of comparative peptide stability in FBS showed no advantage of INGAP-19 over INGAP-P, as both peptides degraded at a similar rate (FIG. 20).

To explore whether efficiency of INGAP-19 could be further augmented by increasing its stability, cyclization (disulfide, head-to-tail, by addition of terminal cysteines) was chosen, as this is a widely used approach to increase peptide stability (Adessi, C. and Soto, C., Curr Med Chem, 2002, 9: 963-978). INGAP-P was similarly cyclized and the new analogs were termed INGAP-19C and INGAP-PC (“C” for cyclized).

Stabilities of INGAP-P, INGAP-PC, INGAP-19 and INGAP-19C were compared in time-course studies of in vitro incubation in FBS. Data showed that INGAP-19C appeared more stable than linear 15-mer (INGAP-P) or 19-mer (INGAP-19) peptides (FIGS. 20, 21,). Importantly, INGAP-19C was equipotent to linear 19-mer (INGAP-19) and had a higher molar efficiency than INGAP-P (FIG. 22) based on studies of Erk½ activation in RINm5 F cells. Additionally, we demonstrated that cyclization alone did not increase activity of INGAP-P (FIG. 22A).

Thus, the results indicate that a 19-mer of rINGAP, INGAP¹⁰²⁻¹²⁰ (SEQ ID NO:4) and a cyclized 19-mer of rINGAP, cyclized INGAP¹⁰²⁻¹²⁰, are more bioactive than INGAP-P 15-mer. Cyclized INGAP¹⁰²⁻¹²⁰ shows greater stability than INGAP-P.

Accordingly, there is provided herein a 19-mer peptide of INGAP, INGAP¹⁰²⁻¹²⁰ (also referred to herein as “INGAP-19 ”, “19-mer”. “19-mer seq 3 ” and SEQ ID NO:4). There is also provided herein a cyclized 19-mer peptide of INGAP, INGAP¹⁰²⁻¹²⁰ (also referred to herein as “INGAP-19C” and SEQ ID NO:6). It is shown herein that 19-mer peptides possess β-cell neogenic and insulin-potentiating activities of INGAP and/or improved stability compared to INGAP-P, indicating 19-mer INGAP peptides as potential novel therapeutics for diabetes.

In an embodiment, there is provided herein an INGAP peptide comprising the sequence set forth in SEQ ID NO: 4 or SEQ ID NO:6. In another embodiment, there is provided herein an INGAP peptide consisting of the sequence set forth in SEQ ID NO: 4 or 6.Compositions and methods of use thereof are also provided.

As used herein, “β-cells” refer to the fully differentiated insulin-producing β-cells of the islets of Langerhans in the pancreas. Pancreatic β-cells are characterized by their secretion of insulin and typically by their cell surface expression of the islet amyloid polypeptide (IAPP).

It should also be understood that analogs, homologs, fragments and variants of 19-mer peptides of the invention which retain biological activities of INGAP are encompassed by peptides of the invention. In an embodiment, variants of SEQ ID NO:4 and SEQ ID NO: 6 are provided having at least 80%, at least 85%, at least 90%, at least 95%, at least 98% or at least 99% sequence identity to SEQ ID NO:4 and SEQ ID NO:6. In another embodiment, variants have at least 80%, at least 85%, at least 90%, at least 95%, at least 98% or at least 99% identity to SEQ ID NO:4 and SEQ ID NO:6 and retain typtophan residues at INGAP positions 103 and 120 (in other words, tryptophan residues at INGAP positions 103 and 120 are not removed, substituted or altered). In yet other embodiments, variants retain at least one of the tryptophan residues at positions 103 and 120 or both tryptophan residues.

Peptides and compositions of the invention can be used for treating or preventing conditions or diseases of the pancreas. Non-limiting examples of such conditions or diseases include metabolic disorders, or conditions such as Type 1 and Type 2 diabetes mellitus, complications of diabetes (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, edema and hyperuricaemia.

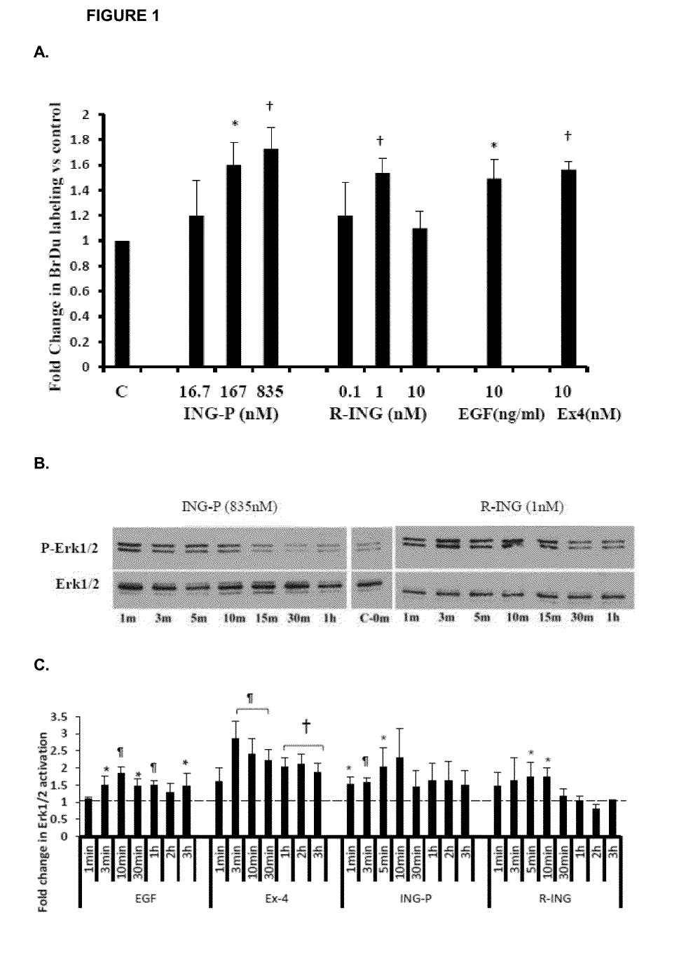

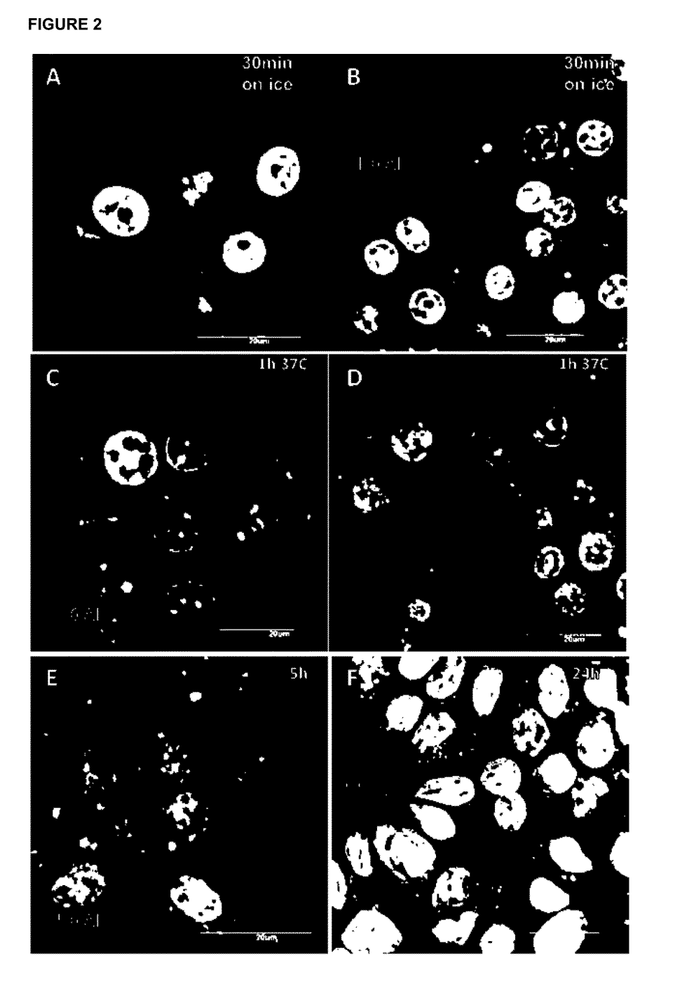

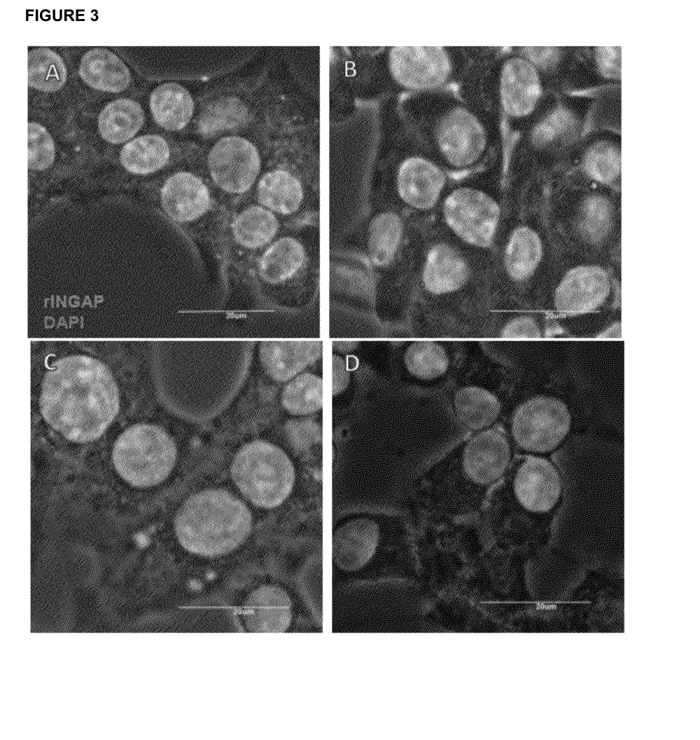

Expansion of β-cell mass can involve several processes, including proliferation of existing islet cells, neogenesis from duct-associated precursors or regeneration of islet cells from dedifferentiated endocrine cells. We show herein that INGAP-P induces: (1) proliferation and endocrine differentiation of normal human pancreatic duct cells (HPDE) (FIGS. 25-29, 34); (2) regeneration of functional islet-like structures from dedifferentiated human islet-derived duct-like structures (DLS) (FIGS. 23, 30-33); and (3) proliferation of RINm5 F cells, an insulin-producing rat cell line (FIGS. 1, 8, 18, 22). It has also been shown that in animal models, INGAP-P can lead to a significant increase in number of pancreatic islet cells and to production of more insulin (U.S. patent application publication no. 2004/0132644). Newly formed β-cells appeared in the wall of, and budding from, pancreatic ducts. These insulin-positive cells resulted from ductal epithelial cell differentiation and islet cell growth, and their appearance was proportional to dose and duration of treatment with INGAP-P. Over longer periods of treatment, these cells migrated away from the duct and formed islets in the parenchyma of the pancreas. After 10 consecutive days of INGAP-P administration, there was a 30% increase in islet number, and by 30 days there was a doubling of the number of islets in the tissues. Similar effects are expected for peptides disclosed herein.

Accordingly, in an embodiment peptides and compositions of the invention promote, enhance or induce β-cell neogenesis. For example, peptides and compositions of the invention improve or restore functionality of pancreatic cells, and/or may increase the number or size of pancreatic β-cells. In another embodiment, peptides and compositions of the invention promote, enhance or induce regeneration of pancreatic β-cells. In another embodiment, peptides and compositions of the invention promote, enhance or induce proliferation of pancreatic β-cells. In yet another embodiment, peptides and compositions of the invention have insulin-potentiating activities. In a further embodiment, peptides and compositions of the invention improve glucose homeostasis in a subject having Type 1 or Type 2 diabetes.

As used herein, “insulin-potentiating activity” and “insulin potentiation” refer to ability to achieve a therapeutic outcome at lower doses of insulin when insulin is administered in combination with peptides or compositions of the invention, compared to administration of insulin alone. In other words, less externally provided insulin is needed to achieve a certain therapeutic outcome when insulin is administered in combination with peptides or compositions of the invention; in the presence of peptides or compositions of the invention, a similar therapeutic outcome is achieved with lower doses of insulin as with higher doses of insulin alone.

In further embodiments, peptides and compositions of the invention prevent β-cell death by, e.g., apoptosis or necrosis of pancreatic β-cells; induce differentiation of new functional islets from primitive duct-like structures (DLS) derived from dedifferentiated adult islets; enhance endocrine differentiation; induce islet cell regeneration from cells associated with ductal epithelium, leading to new islet formation; and/or lead to reversal of hyperglycemia. In a particular embodiment, peptides and compositions of the invention induce differentiation of pancreatic duct cells, and/or allow such cells to avoid apoptotic pathways.

In still further embodiments, peptides of the invention have better in vitro stability, greater stability in the circulation and/or a longer half-life in vivo compared to INGAP-P 15-mer peptide.

In an embodiment, a β-cell associated disorder is treated or prevented by peptides and compositions of the invention. In a particular embodiment, diabetes, particularly Type 1 diabetes, Type 2 diabetes, preclinical Type 1 diabetes, and/or diabetic complications are treated or prevented by peptides and compositions of the invention.

Thus, in one aspect there is provided herein a method for treating or preventing a metabolic disorder in a subject in need thereof, comprising administering a therapeutically-effective amount of a peptide or composition of the invention to the subject. In another aspect, there is provided a method for treating or preventing diabetes in a subject in need thereof, comprising administering a therapeutically-effective amount of a peptide or composition of the invention, e.g. SEQ ID NO:4, to the subject.

In yet another aspect, there is provided a method for preventing degeneration of pancreatic β-cells and/or for improving and/or restoring functionality of pancreatic β-cells in a subject in need thereof, comprising administering a therapeutically-effective amount of a peptide or composition of the invention to the subject. In one aspect, the number or size of pancreatic cells, e.g. β-cells, is increased in the subject, and/or plasma insulin levels are increased in the subject, and/or glucose homeostasis is restored or improved in the subject.

In a further aspect, there is provided a method of protecting islet cells against diabetogenic agents in vitro and/or in vivo, comprising contacting an eukaryotic cell with, or administering to a subject, a peptide or composition of the invention. In an embodiment, islet viability is improved, and/or islet dysfunction is blocked, and/or β-cell mass is preserved in a subject after administration of a peptide or composition of the invention.

According to another embodiment of the invention, a method of inducing differentiation of β-cell progenitors is provided, comprising: contacting a culture of pancreatic duct cells comprising β-cell progenitors with a preparation of a peptide of the invention, to induce differentiation of said β-cell progenitors. In an embodiment, pancreatic duct cells of a mammal with pancreatic endocrine failure can be removed from the body and treated in vitro. Duct cells typically comprise β-cell progenitors. Thus treatment with a preparation of a peptide of the invention will induce differentiation of the β-cell progenitors. Cells treated with peptides of the invention can then be used as an autologous transplant into the mammal from which they were derived. Such an autologous treatment minimizes adverse host versus graft reactions involved in transplants.

In one embodiment, the subject can be a rodent, a canine, a pig, a primate or a human. Although methods of the present invention can be used in any mammal, the subject is preferably a human.

The term “homolog” is used to mean those amino acid or nucleic acid sequences which have slight or inconsequential sequence variations from the sequences of the peptides described herein, such that homolog sequences function in substantially the same manner as the original sequences. Sequence variations may be attributable to local mutations or structural modifications. Sequences having substantial sequence identity include nucleic acid sequences having at least 80%, at least 85%, at least 90%, at least 95%, at least 98% or at least 99% sequence identity to sequences that encode peptides as provided herein, or amino acid sequences having at least 80%, at least 85%, at least 90%, at least 95%, at least 98% or at least 99% sequence identity to peptides provide herein (such as SEQ ID NO: 4 or SEQ ID NO:6). Sequence identity can be calculated according to methods known in the art. Nucleic acid sequence identity is most preferably assessed by the algorithm of BLAST version 2.1 advanced search. A series of programs is available at http://www.ncbi.nlm.nih.gov/BLAST.

The term “analog” is used to mean an amino acid or nucleic acid sequence which has been modified as compared to the sequence of the peptides described herein, wherein the modification does not alter biological activity of the sequence (e.g., induction of pancreatic β-cell neogenesis, induction of pancreatic β-cell regeneration, improvement of glucose homeostasis, or reversal of hyperglycemia) as described herein. Modified sequences or analogs may have improved properties over peptides described herein, e.g., SEQ ID NO:4 or SEQ ID NO:6.

Also encompassed are sequences that hybridize to the complement of a nucleotide sequence encoding a peptide of the invention, and that hybridize to the complement of a nucleotide sequence encoding a peptide which maintains a biological activity of SEQ ID NO:4 or SEQ ID NO:6, e.g., β-cell neogenesis activity, in vivo stability, etc. The term “sequence that hybridizes” means a nucleic acid sequence that can hybridize to a sequence under stringent hybridization conditions. Appropriate “stringent hybridization conditions” which promote DNA hybridization are known to those skilled in the art, and may be found for example in Current Protocols in Molecular Biology, John Wiley & Sons, N.Y. (1989), 6.3.1 -6.3.6. The term “stringent hybridization conditions” as used herein means that conditions are selected which promote selective hybridization between two complementary nucleic acid molecules in solution. Hybridization may occur to all or a portion of a nucleic acid sequence molecule. The hybridizing portion is at least 50% the length with respect to one of the polynucleotide sequences encoding a polypeptide. In this regard, the stability of a nucleic acid duplex, or hybrids, is determined by the Tm, which in sodium containing buffers is a function of the sodium ion concentration, G/C content of labeled nucleic acid, length of nucleic acid probe (1), and temperature (Tm=81.5° C.-16.6 (Log10 [Na+])+0.41 (%(G+C)−600/1). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a greater than 95% identity, the final wash will be reduced by 5° C. Based on these considerations, in one embodiment stringent hybridization conditions are defined as: hybridization at 5×sodium chloride/sodium citrate (SSC)/5 × Denhardt's solution/1.0% SDS at Tm (based on the above equation) −5° C., followed by a wash of 0.2×SSC/0.1% SDS at 60° C.

Peptides may be modified to contain amino acid substitutions, insertions and/or deletions that do not alter biological activity of the peptide. Conservative amino acid substitutions involve replacing one or more amino acids of a peptide with amino acids of similar charge, size, and/or hydrophobicity characteristics. When only conservative substitutions are made, it is expected that a resulting analog would be functionally equivalent to an unsubstituted peptide. Non-conservative substitutions involve replacing one or more amino acids of a peptide with one or more amino acids which possess dissimilar charge, size, and/or hydrophobicity characteristics.

A peptide may be modified to make it more therapeutically effective or suitable, e.g., stable. For example, a peptide of the present invention may be converted into a pharmaceutically-acceptable salt by reacting with inorganic acids such as, for example, hydrochloric acid, sulphuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and tolunesulphonic acids, for example. Pharmaceutically-acceptable salts are well-known in the art and pharmaceutically-acceptable salts of peptides and analogs, homologs, fragments and variants thereof are encompassed herein.

Additionally, peptides may be chemically modified by covalent conjugation to a polymer to increase its circulating half-life, for example. Exemplary polymers, and methods to attach them to peptides, are shown in U.S. Pat. Nos. 4,766,106, 4,179,337, 4,495,285, and 4,609,546. Non-limiting examples of polymers are polyoxyethylated polyols and polyethylene glycol (PEG). PEG is soluble in water at room temperature and has the general formula: R(O—CH₂—CH₂)ₙO—R where R can be hydrogen, or a protective group such as an alkyl or alkanol group. In an embodiment, the protective group has between 1 and 8 carbons, or is methyl. The symbol n is a positive integer, for example between 1 and 1,000, or between 2 and 500. In an embodiment, the PEG has an average molecular weight between 1000 and 40,000, between 2000 and 20,000, or between 3,000 and 12,000. PEG may have at least one hydroxy group, or a terminal hydroxy group. This hydroxy group may be activated to react with a free amino group on the inhibitor.

The present invention also provides expression vectors comprising a nucleic acid sequence encoding a peptide of the invention or a fragment or analog thereof

Possible expression vectors include, but are not limited to, cosmids, plasmids, artificial chromosomes, viral vectors or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, operatively linked to the nucleic acid molecule of the invention. “Operatively linked” is intended to mean that the nucleic acid is linked to regulatory sequences in a manner which allows expression of the nucleic acid.

There is provided herein a recombinant expression vector containing a nucleic acid molecule of the invention, or a fragment or analog thereof, and necessary regulatory sequences for transcription and translation of the inserted peptide-encoding sequence.

Suitable regulatory sequences may be derived from a variety of sources, including bacterial, fungal, viral, mammalian, or insect genes (for example, see the regulatory sequences described in Goeddel, Gene Expression Technology: Methods in Enzymology 185, Academic Press, San Diego, Calif. (1990). Selection of appropriate regulatory sequences is dependent on the host cell, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

Recombinant expression vectors of the invention may also contain a selectable marker gene which facilitates selection of host cells transformed or transfected with a peptide of the disclosure. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin, such as IgG. Transcription of a selectable marker gene is monitored by changes in concentration of the selectable marker protein such as β-galactosidase, chloramphenicol acetyltransferase, or firefly luciferase. If a selectable marker gene encodes a protein conferring antibiotic resistance such as neomycin resistance, transformant cells can be selected with G418.Cells that have incorporated a selectable marker gene will survive, while other cells die. This makes it possible to visualize and assay for expression of recombinant expression vectors of the disclosure and in particular to determine the effect of a mutation on expression and phenotype. It will be appreciated that selectable markers can be introduced on a separate vector from the nucleic acid of interest.

Recombinant expression vectors provided herein may also contain genes which encode a moiety which provides increased expression of a peptide; increased solubility of a recombinant peptide; and/or aid in purification of a target recombinant peptide by acting as a ligand in affinity purification. For example, a proteolytic cleavage site may be added to a target recombinant peptide to allow separation of a recombinant protein from a fusion moiety subsequent to purification of a fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to a recombinant peptide.

Recombinant expression vectors can be introduced into host cells to produce a transformed host cell. The term “transformed host cell” is intended to include cells that are capable of being transformed or transfected with a recombinant expression vector of the invention. The terms “transduced”, “transformed with”, “transfected with”, “transformation” and “transfection” are intended to encompass introduction of nucleic acid (e.g. a vector or naked RNA or DNA) into a cell by one of many possible techniques known in the art. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation, microinjection, RNA transfer, DNA transfer, artificial chromosomes, viral vectors and any emerging gene transfer technologies. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, peptides of the disclosure may be expressed in yeast cells or mammalian cells. Other suitable host cells can be found in Goeddel, Gene Expression Technology: Methods in Enzymology 185, Academic Press, San Diego, Calif. (1991). In addition, peptides of the disclosure may be expressed in prokaryotic cells, such as Escherichia coli (Zhang et al., Science 303 (5656): 371-3 (2004)).

Mammalian cells suitable for use in methods described herein include, among others: COS (e.g., ATCC No. CRL 1650 or 1651), BHK (e.g. ATCC No. CRL 6281), CHO (ATCC No. CCL 61), and HeLa (e.g., ATCC No. CCL 2) and 3 T3 mouse fibroblasts (e.g. ATCC No. CCL92).

Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include without limitation pCDM8 (Seed, B., Nature 329:840 (1987)), pMT2 PC (Kaufman et al., EMBO J. 6:187-195 (1987)) and pCMV (Clontech, California, U.S.A.).

Alternatively, peptides of the invention may also be expressed in non-human transgenic animals, such as rats, mice, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866). The present invention also encompasses tissues and cells derived or isolated from such animals.

In addition to analogs and homologs described above, in certain embodiments, peptides of the invention may further be recombinantly fused to a heterologous polypeptide at the N- or C-terminus or chemically conjugated (including covalent and non-covalent conjugations) to polypeptides or other compositions. For example, peptides may be recombinantly fused or conjugated to molecules useful as labels in detection assays and effector molecules such as heterologous polypeptides, histidine (HIS) tags, drugs, radionuclides, or toxins. See, e.g., PCT publications WO 92/08495; WO 91/14438; WO 89/12624; U.S. Pat. No. 5,314,995; and EP 396,387. Any type of molecule may be covalently attached to peptides of the invention as long as it does not inhibit biological activity of the peptide. For example, but not by way of limitation, peptide derivatives include peptides that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc.

The heterologous polypeptide to which a peptide is fused may be useful for example to increase the in vivo half life of the peptide, or for use in immunoassays using methods known in the art. Peptides of the invention can be fused to marker sequences, such as a polypeptide to facilitate purification or detection. In general, it should be understood that peptides of the present invention may be used in non-conjugated form or may be conjugated to at least one of a variety of molecules, e.g., to improve therapeutic properties of the molecule, to improve pharmacokinetic properties of the molecule, etc.

In certain embodiments, a peptide of the invention includes an additional amino acid sequence or one or more moieties. Exemplary modifications are described in more detail below. For example, peptides may be modified to add an additional functional moiety (e.g., PEG, a drug, a toxin, an imaging agent or a label).

Furthermore, nucleotide or amino acid substitutions, deletions, or insertions leading to conservative substitutions or changes at “non-essential” amino acid regions may be made. For example, a peptide may be identical to the starting sequence except for one or more individual amino acid substitutions, insertions, or deletions, e.g., one, two, three, four, five, six, seven, eight, nine, or ten or more individual amino acid substitutions, insertions, or deletions may be made. In other embodiments, a peptide derived from a starting peptide may be identical to the starting sequence except for one, two or fewer, three or fewer, four or fewer, five or fewer, six or fewer, seven or fewer, eight or fewer, nine or fewer, or ten or fewer individual amino acid substitutions, insertions, or deletions. In certain embodiments, a peptide derived from a starting peptide has one, two, three, one to two, one to three, one to five or one to ten individual amino acid substitutions, insertions, or deletions relative to the starting sequence. In a particular embodiment, at least one or both of the tryptophan residues at positions 2 and 19 of SEQ ID NO:4 are retained in a derivative peptide, i.e., at least one or both of the tryptophan residues at positions 2 and 19 are retained.

Also encompassed in the present invention are fragments, derivatives, modifications, or variants of peptides described herein, as well as analogs and homologs described above, and any combination thereof. The terms “fragment,” “variant,” “derivative”, “modification”, “homolog” and “analog” when referring to peptides of the present invention include any polypeptides which retain at least some of the biological activities of the corresponding starting peptide sequences. The terms “variant,” “derivative” and “modification” are used interchangeably herein.

Variants of peptides of the present invention include fragments, polypeptides with altered amino acid sequences due to amino acid substitutions, deletions, or insertions as described herein, and modifications as described herein. Variant polypeptides may comprise conservative or non-conservative amino acid substitutions, deletions or additions as described herein. Variants may also have one or more residues chemically derivatized by reaction of a functional side group. Also included as variants are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. Additionally, a variant may contain one or more non-classical amino acids.

Thus in one embodiment, analogs, homologs, fragments or variants of peptides disclosed herein are encompassed by the present invention. In an embodiment, analogs, homologs, fragments or variants retain biological activity/activities of the starting peptide, e.g., β-cell neogenesis activity, insulin potentiating activity, ability to restore or improve glucose homeostasis in a subject, ability to reverse hyperglycemia, binding to cellular receptors, stability, etc. One or more of the biological activities of a peptide may be retained by analogs, homologs, fragments or variants. In an embodiment, an analog, homolog, fragment or variant retains at least one biological activity or property of the starting peptide.

In an embodiment, peptides of the invention are purified, or substantially pure. In another embodiment, peptides of the invention are synthesized chemically.

### Pharmaceutical Compositions and Methods of Administration

Pharmaceutical compositions encompassing peptides of the invention are encompassed herein. Peptides of the present invention can be administered to a subject in a conventional dosage form prepared by combining a peptide of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables.

Methods of preparing and administering peptides or analogs, homologs, fragments or variants thereof to a subject are well-known in the art or are readily determined by those skilled in the art. The route of administration of peptides and compositions of the invention may be, for example, oral, parenteral, by inhalation or topical. The term parenteral as used herein includes, e.g., intravenous, intraarterial, intraperitoneal, intramuscular, subcutaneous, rectal or vaginal administration. In a particular embodiment, a peptide or composition of the invention is administered by injection. In an embodiment, the administration route is intravenous. In another embodiment, a peptide or composition of the invention is administered orally, e.g., once daily, twice daily, or three times daily.

Usually, a suitable pharmaceutical composition for injection may comprise a buffer (e.g. acetate, phosphate or citrate buffer), a surfactant (e.g. polysorbate), and optionally a stabilizer agent (e.g. human albumin), etc. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1 M and preferably 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for extemporaneous preparation of sterile injectable solutions or dispersions. In such cases, a composition must be sterile and should be fluid to the extent that easy syringability exists. It should be stable under conditions of manufacture and storage and will preferably be preserved against contaminating action of microorganisms, such as bacteria and fungi. A carrier can be a solvent or dispersion medium containing, for example, water, ethanol, polyol (e.g., glycerol, propylene glycol, and liquid polyethylene glycol, and the like), and suitable mixtures thereof. Proper fluidity can be maintained, for example, by use of a coating such as lecithin, by maintenance of required particle size in the case of dispersion and by use of surfactants. Suitable formulations for use in therapeutic methods disclosed herein are described in Remington's Pharmaceutical Sciences, Mack Publishing Co., 16th ed. (1980).

Prevention of action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in a composition. Prolonged absorption of injectable compositions can be brought about by including in a composition an agent which delays absorption, for example, aluminum monostearate and gelatin.

In any case, sterile injectable solutions can be prepared by incorporating a peptide of the invention (by itself or in combination with other active agents) in a required amount in an appropriate solvent with one or a combination of ingredients, as required and easily determined by a person of skill in the art, followed by filtered sterilization. Generally, dispersions are prepared by incorporating an active compound into a sterile vehicle, which contains a basic dispersion medium and the required other ingredients from those enumerated above. In the case of sterile powders for preparation of sterile injectable solutions, preferred methods of preparation are vacuum drying and freeze-drying, which yields a powder of an active ingredient plus any additional desired ingredient from a previously sterile-filtered solution thereof Preparations for injections are processed, filled into containers such as ampoules, bags, bottles, syringes or vials, and sealed under aseptic conditions according to methods known in the art.

After a liquid pharmaceutical composition is prepared, it may be lyophilized to prevent degradation and to preserve sterility. Methods for lyophilizing liquid compositions are known to those of ordinary skill in the art. Just prior to use, a composition may be reconstituted with a sterile diluent (Ringer's solution, distilled water, or sterile saline, for example) which may include additional ingredients. Upon reconstitution, a composition is administered to subjects using those methods that are known to those skilled in the art.

Further, preparations may be packaged and sold in the form of a kit. Such articles of manufacture will preferably have labels or package inserts providing instructions for use and may have additional components required for use of preparations.

Those skilled in the art will appreciate that effective doses of peptides and compositions of the present invention, e.g. for preventing or treating diabetes, vary depending upon many different factors, including means of administration, characteristics or physiological state of the subject (such as state of health), other medications being administered, whether treatment is diagnostic, prognostic, prophylactic or therapeutic, and so on. Dosage may be determined using routine methods known to those of skill in the art in order to optimize safety and efficacy.

Clearly, an amount of a fusion peptide to be administered will also depend on the subject to which it is to be administered. In the case where the subject is a human, amount of a peptide to be administered will depend on a number of factors including the age of the patient, the severity of the condition and the past medical history of the patient and always lies within the sound discretion of the administering physician. Generally, a total daily dose of peptides of the invention administered to a human or other mammal in single or in divided doses can be in amounts, for example, of from 0.1 mg/Kg/day to 30 mg/Kg/day of the peptide, from 0.1 mg/Kg/day to 20 mg/Kg/day of the peptide, or from 2 mg/Kg/day to 10 mg/Kg/day of the peptide, in single or multiple doses. Single dose compositions may contain such amounts or submultiples thereof to make up a daily dose. In an embodiment, 5 mg/kg is given daily, intraperitoneally (IP).

Dosing regimens and formulations of INGAP peptide have been described (see, e.g., US application publication no. 2004/0132644).

In an embodiment, peptides of the invention are formulated or used in a pharmaceutically acceptable salt form. In a particular embodiment, the pharmaceutically acceptable salt is an acetate salt.

In an embodiment, peptides of the invention are substantially pure.

Stability may be determined using methods known in the art. For example, stability of peptides is determined by comparing various parameters including, but not limited to, degree of purity, total percentage of impurities, percentage of individual impurities (as determined by HPLC or other suitable quantitative method), appearance, and water content of a sample. An HPLC method can be used to determine any increase in levels of degradation products relative to levels of the therapeutic peptide.

Peptide samples, whether in solution or a lyophilized powder, may be stored at various temperatures, in the presence or absence of humidity, and in light or dark vials. Degradation during different storage conditions can lead to an increase in impurities and a decrease in therapeutic peptide content. In some embodiments, it is desirable that a sample preparation is more than 80% pure, more than 90% pure, more than 95% pure, or more than 97% pure.

Peptides of the present invention may also be administered as a component of a pharmaceutically administrable composition. In other words, a peptide may be present in a formulation for administration to a subject in need thereof. An inventive peptide may be the sole active ingredient for, e.g., treatment of diabetes. Alternatively, a composition may also contain one or more additional compounds, e.g., a second agent that may be used to treat the same or related conditions.

It should be understood that peptides of the invention can optionally be administered in combination with other agents that are effective in treating the disorder or condition in need of treatment. In keeping with the scope of the present disclosure, peptides and compositions of the invention may be used with other therapeutic or prophylactic agents. Peptides of the invention may be administered concomitantly or sequentially with a second agent. It should be understood that any therapeutic agent for Type 1 or Type 2 diabetes or related disorders is contemplated for use in combination with peptides of the invention. Examples of such therapeutic or prophylactic agents include, without limitation, antidiabetic agents such as metformin, sulphonylureas (e.g. glibenclamide, tolbutamide, glimepiride), nateglinide, repaglinide, thiazolidinediones (e.g. rosiglitazone, pioglitazone), PPAR-gamma-agonists (e.g. GI 262570) and antagonists, PPAR-gamma/alpha modulators (e.g. KRP 297), alpha-glucosidase inhibitors (e.g. acarbose, voglibose), DPPIV inhibitors (e.g. LAF237, MK-431), alpha2-antagonists, agents for lowering blood sugar, cholesterol-absorption inhibitors, HMGCoA reductase inhibitors (such as a statin), insulin and insulin analogues, GLP-1 and GLP-1 analogues (e.g. exendin-4) and/or amylin. In an embodiment, peptides and compositions of the invention are used in combination with the immune modulator anakinra, an IL-1 inhibitor approved for treatment of rheumatoid arthritis, but with evidence of efficacy in diabetes.

In one embodiment, a second therapeutic agent is an agent which preserves β-cell mass, for example by blocking cell death or apoptosis of β-cells, protecting islets against detrimental effects of IL-1, e.g., IL-1β, protecting against diabetogenic agents, and/or otherwise protecting or improving islet viability and/or function. A second therapeutic agent may also reverse insulin resistance, control intestinal glucose absorption, normalise hepatic glucose production, and/or improve beta-cell glucose sensing and insulin secretion. In one embodiment, a second therapeutic agent may be an inhibitor of the transcription factor NF-κB, or an inhibitor of the cytokine-induced activation of the transcription factor NF-κB. In an embodiment, a second therapeutic agent is anakinra In other embodiments, a second therapeutic agent is insulin, an insulin analogue, an SGLT 2 inhibitor, a new islet formation induces, a stem cell therapy, a T-lymphocyte inhibitor, an IL 12 activator, a STAT 4 activator, an immune modulator, an islet implant, an anti-inflammatory agent, an anti-CD3 monoclonal antibody, and/or an interleukin-1 (IL-1) receptor antagonist.

### EXAMPLES

The present invention will be more readily understood by referring to the following examples, which are provided to illustrate the invention and are not to be construed as limiting the scope thereof in any manner.

### Example 1

### INGAP-P and r-INGAP Dose-Dependently Increase Proliferation of RIN-m5 F Cells

Although pancreatic ductal cells have been understood to be a particular target of INGAP (Rosenberg, L., et al., 1988, Diabetes, 37: 334-341; Pittenger, G. L., et al., 2007, Pancreas, 34: 103-111), a number of studies including results of clinical trials suggest that β-cells are also responsive to INGAP stimulation. To study effects of INGAP on β-cells we used RIN-m5 F, a rat insulinoma cell line, commonly used as a β-cell surrogate in vitro (Cozar-Castellano, I., et al., 2008, Diabetes, 57: 3056-3068). Although no significant effect on insulin expression was observed in our experiments, the data showed that both INGAP-P and r-INGAP dose dependently induced BrdU incorporation in RIN-m5 F cells after 24 h (FIG. 1A), with the most effective concentrations being 1 nM for r-INGAP (1.5× increase compared to negative control, which was treatment with PBS (equal to 1)) and 835 nM for INGAP-P (1.8× increase compared to negative control). Overall, this fold change was consistent with earlier data on hamster ductal explants and ARIP cells (Rafaeloff, R., et al., 1997, J Clin Invest, 99: 2100-2109). Similar mitogenic effects were observed with EGF (10 ng/ml, 1.49× increase) and Exendin 4 (10 nM, 1.56× increase), which were used as positive controls (FIG. 1A).

Increase in BrdU incorporation was consistent with a rapid temporal activation of Erk½, observed between 1 and 15 min after addition of either r-INGAP or INGAP-P (FIG. 1B, C). To note, EGF and Ex-4 appeared to generate a longer lasting Erk½ activation (FIG. 1C), which suggests differences in signaling pathways activated by these factors and INGAP. These results showed that both protein and peptide acted in a similar manner but with different molar efficiencies (a difference of at least 167 fold). It should be noted that a similar difference in efficiency was observed between the 15-mer peptide and the rINGAP protein using a model of in vitro regeneration of functional human islets from dedifferentiated, islet-derived duct-like structures (Assouline-Thomas, B., et al., 2010, Protein Expr Purif, 69: 1-8), and in HPDE cells (Beatrice, G. and Assouline-Thomas, L. R., Islet neogenesis associated protein (INGAP) induces endocrine differentiation of human pancreatic ductal cells in vitro. 70th American Diabetes Association Meeting. 2009). The most likely explanation of this phenomenon is that INGAP protein and INGAP-P interact differently with the cell surface and/or activate different signalling pathways.

INGAP-19 and INGAP-19C peptides were also shown to induce Erk½ activation in RINm5F cells, and both were more effective than INGAP-P or INGAP-PC (FIG. 23).

### Example 2

### r-INGAP and INGAP¹⁰²⁻¹²⁰ Bound RIN-m5 F Cells form Cluster-Like Complexes on the Cell Surface, Whereas INGAP¹⁰⁴⁻¹¹⁸ Rapidly Internalizes into the Cytoplasm

To determine how INGAP binds and internalizes into RIN-m5 F cells, we used r-INGAP labeled with fluorescent reactive dyes DyLight −488 (green) and −594 (red) and 5-FAM-labeled INGAP-P. As shown in FIG. 10, 50n M DyLight-488 r-INGAP bound the cell surface of RIN-m5 F cells within minutes and formed small clusters and patches on the cell surface, resembling the crosslinking of membrane multiprotein complexes described for other ligands. This was observed both at 37° C. and on ice, which suggests high affinity receptor binding. This is different from a homogeneous staining exhibited by Cholera Toxin B (CTB, AlexaFluor 594) and Transferrin (Texas Red, both from Invitrogen) that were used as positive markers for caveolin and clathrin mediated endocythosis (FIG. 2A, B). Although first signs of internalization were observed after 15 min (FIGS. 10, 11), the protein appeared to remain clustered on the cell surface for several hours (FIG. 2C-E, FIG. 11), unlike Transferrin and CTB that internalize withing 1 h (FIG. 2, C, D). After a 5 h-incubation, most of the fluorescent label was seen inside cells (FIG. 2E) and was partially co-localized with a lysosomal marker (LysoTracker red). After 24 h, all labeled rINGAP appeared to internalize and to associate with lysosomes, albeit partially, and showed no further binding to the cell surface (FIG. 2F). Results were similar for INGAP¹⁰²⁻¹²⁰ (FIG. 19).

Interestingly, in chase experiments, when cells were exposed to DyLight488 rINGAP only for 1 h, followed by washing and culture without rINGAP for 5 or 24 h, the amount of internalized rINGAP was not significantly lower than after continuous incubation (FIG. 12). This suggested that most INGAP receptors were ligand-bound rather quickly, within 1 h, and that receptor turnover time probably exceeded 24 h.

The lack of co-migration between rINGAP and CTB or Transferrin suggests that rINGAP is not internalized via either a clathrin- or caveolin-mediated pathway. This is in line with the results of immunostaining for clathrin and caveolin, showing no co-localization with rINGAP (FIG. 11). We weren't able to verify these data with specific inhibitors of clathrin-mediated (Chlorpromazin, Dansylcadaverin) or caveolin-mediated endocytosis (Filipin, and β-methylcyclodextrin), as well as Dynasore (dynamin inhibitor) due to the observed cytotoxicity of these drugs, developing faster than rINGAP internalization. We show however, that rINGAP internalization was inhibited by Wortmannin (inhibitor of fluid-phase pinocytosis and PI3K) and by Cytochalasin D (inhibitor of actin polymerization), which is suggestive of macropinocytosis as a major mechanism for rINGAP endocytosis.

In contrast to r-INGAP and INGAP¹⁰²⁻¹²⁰, no accumulation or clustering of FAM-labeled INGAP¹⁰⁴⁻¹¹⁸ was observed on the cell surface (FIG. 4). These results indicated that the 15-mer peptide was internalized as soon as it bound to the cell membrane. Labeled peptide was visible in the cytoplasm of RIN-m5 F cells after 5 min of incubation, reaching a plateau after 30 min (FIG. 4). As seen in FIG. 4C, INGAP-P appears to co-localize with early endosomes after 30 min and then gradually migrates into the lysosomal compartment, co-localizing with LysoTracker red (FIG. 4D, F).

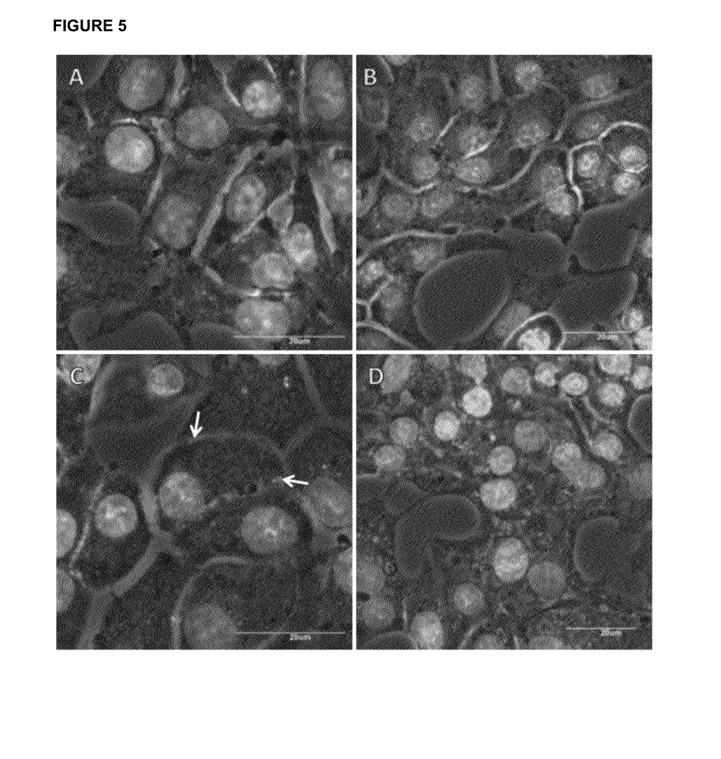

Besides differences in the dynamics of cell binding and internalization, some other differences between protein and peptide have been observed. For example, internalized INGAP-P appears to degrade faster, as shown in 24 h experiments with continuous and “chase” incubations (FIG. 13). Also, internalization of INGAP-P was inhibited on ice or by pre-incubation with the caveolae inhibitor Filipin (FIG. 14), which suggests that this process might be mediated by caveolae/lipid raft endocytosis. Inhibitors of clathrin-dependent endocytosis (Chlorpromazin, Dansylcadaverin) did not have a significant effect (not shown). On the other hand, INGAP-P internalization is inhibited by a 15 min pre-incubation with cytochalasinD, resulting in formation of small clusters on the cell surface (FIG. 5C). This suggests that actin filaments are involved in the process of INGAP-P internalization. However, it's unlikely to be macropinocytosis, as Wortmannin did not appear to have inhibitory effect on this process (FIG. 5D).

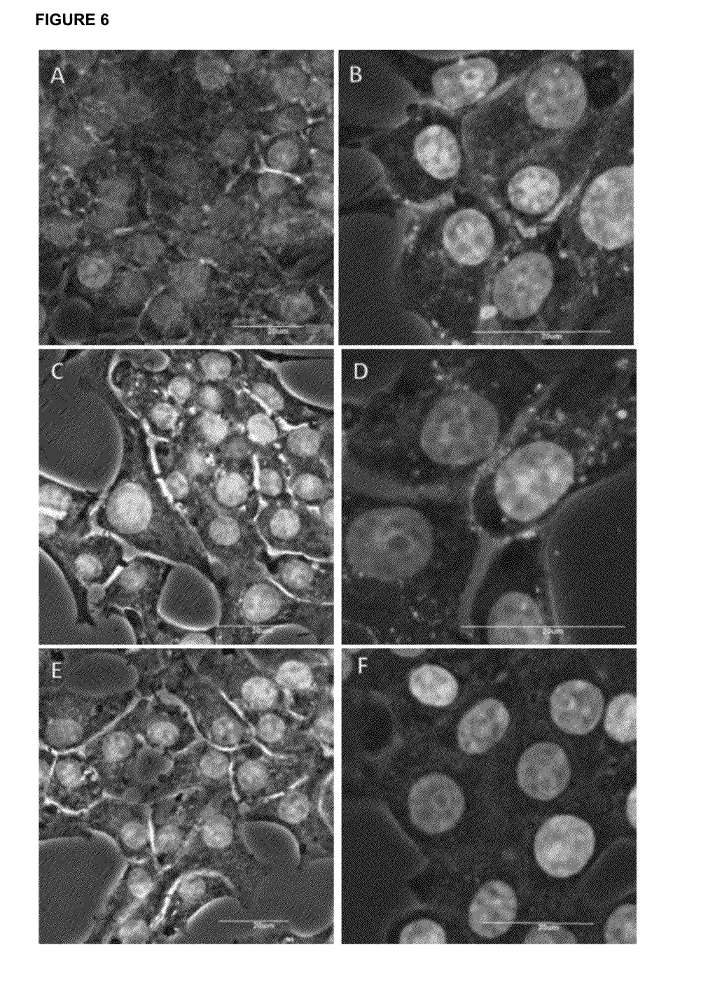

To investigate whether rINGAP, INGAP¹⁰²⁻¹²⁰ and INGAP¹⁰⁴⁻¹¹⁸ act via the same receptor, DyLight488-rINGAP and FAM-INGAP102-120 _{or INGAP}¹⁰⁴⁻¹¹⁸ were used in competition experiments with 20× molar excess of unlabeled protein or peptide. The results showed that internalization of the protein was partially inhibited by unlabeled protein and internalization of peptides was partially inhibited by unlabeled peptides, but they didn't appear to inhibit each other at concentrations tested (FIG. 6). This result suggests that the protein and peptides may not bind the same receptor.

### Example 3

### Erk½ Activation

To compare the potency of 19-mer extended peptides (19-mer seq1, seq2 and seq3; see Table 1) and the 15-mer INGAP-P peptide (see Table 1), Erk½ activation was measured in RINm5 F cells. Results are shown in FIG. 18. Data presented in FIG. 18 show that 19-mer seq3 was 3 times more potent in Erk½ activation in RIN cells, compared to 15-mer INGAP-P peptide and the 2 other 19-mer sequences, when tested at the same concentration. 19-mer seq3 was about 2.5 times more potent at the 1× concentration than the 15-mer peptide at the 10× concentration, suggesting higher efficiency of the 19-mer seq3 peptide.

Activation of Erk½ may be mediated by a number of signaling cascades initiated at the cell membrane level by receptor tyrosine kinases (RTK) or by different classes of G-protein coupled receptors (GPCRs). These signaling cascades include PKC, PKA, PI3 K or Ras/Raf-dependent pathways. Since the nature of the INGAP receptor is unknown, we screened for both RTK and GPCR- initiated signaling events using phospho-specific antibodies and pharmacological inhibitors of the above-mentioned pathways. For comparison we used EGF (10 ng/ml) and Ex- 4 (10 nM), found to be mitogenic for RIN-m5 F cells at the indicated concentrations (FIG. 1A). Because EGF signals through a classical RTK pathway and Ex-4 is an agonist of a G-protein coupled GLP-1 receptor, such a comparison may provide important clues to how INGAP works.

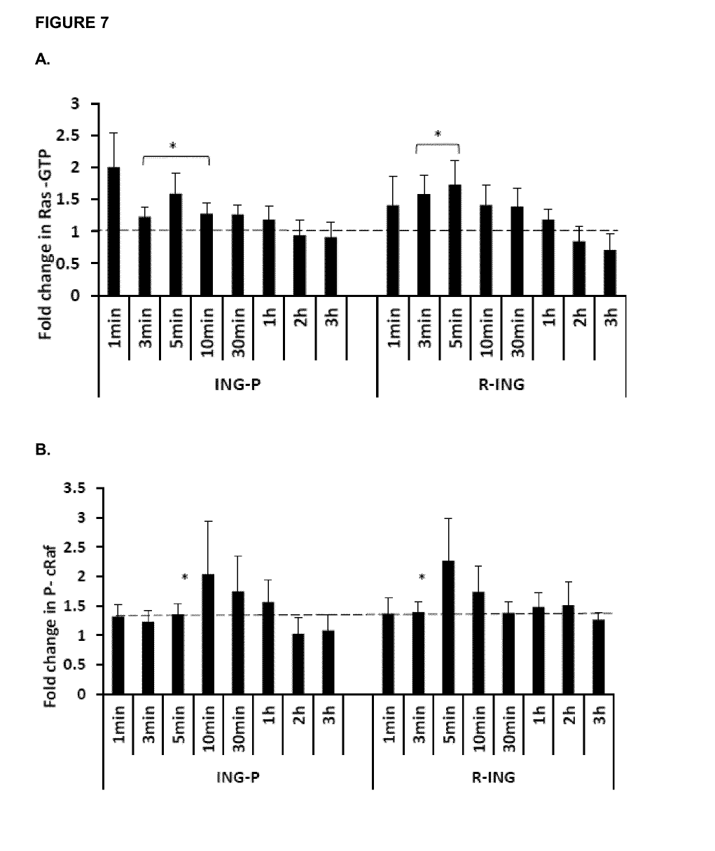

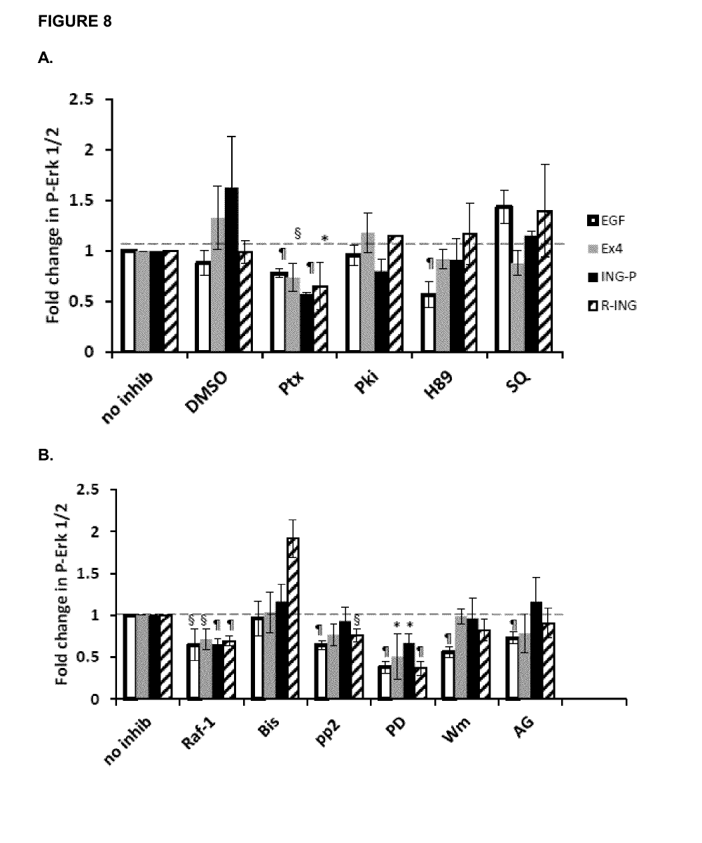

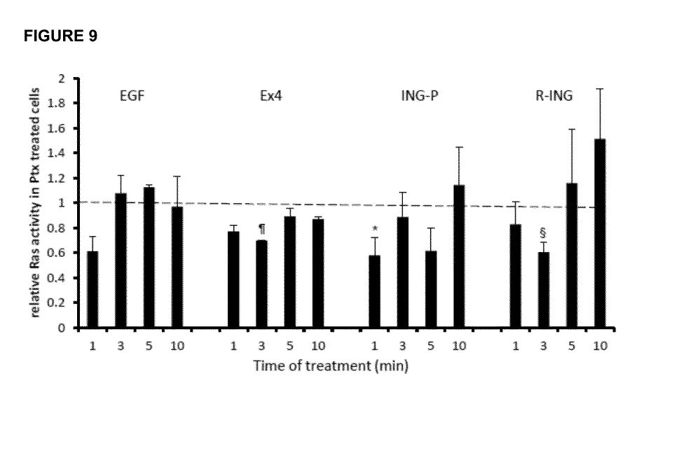

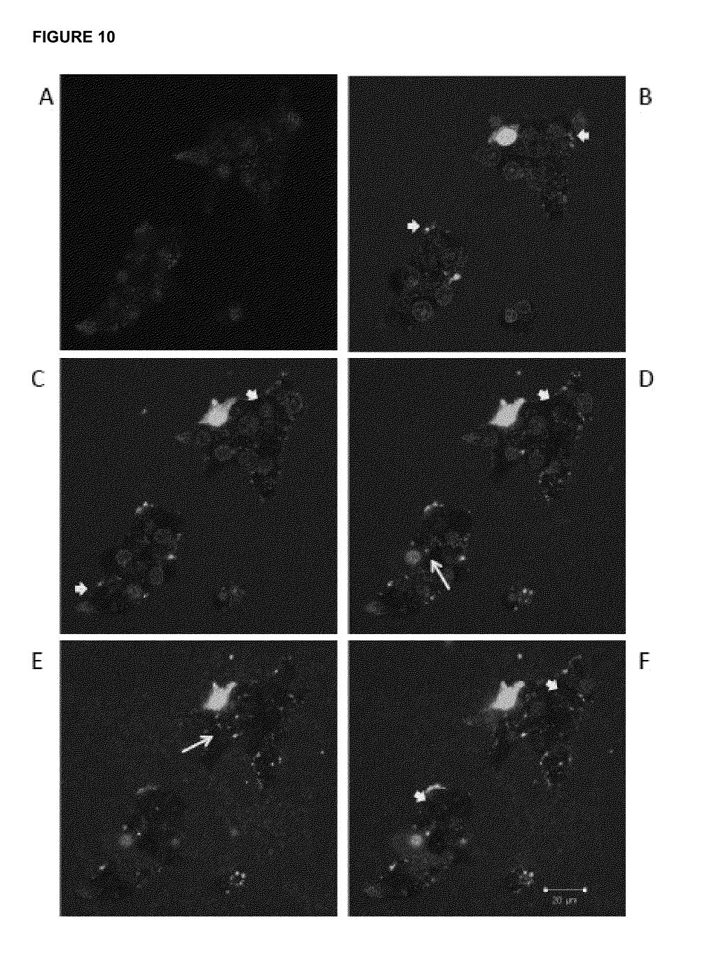

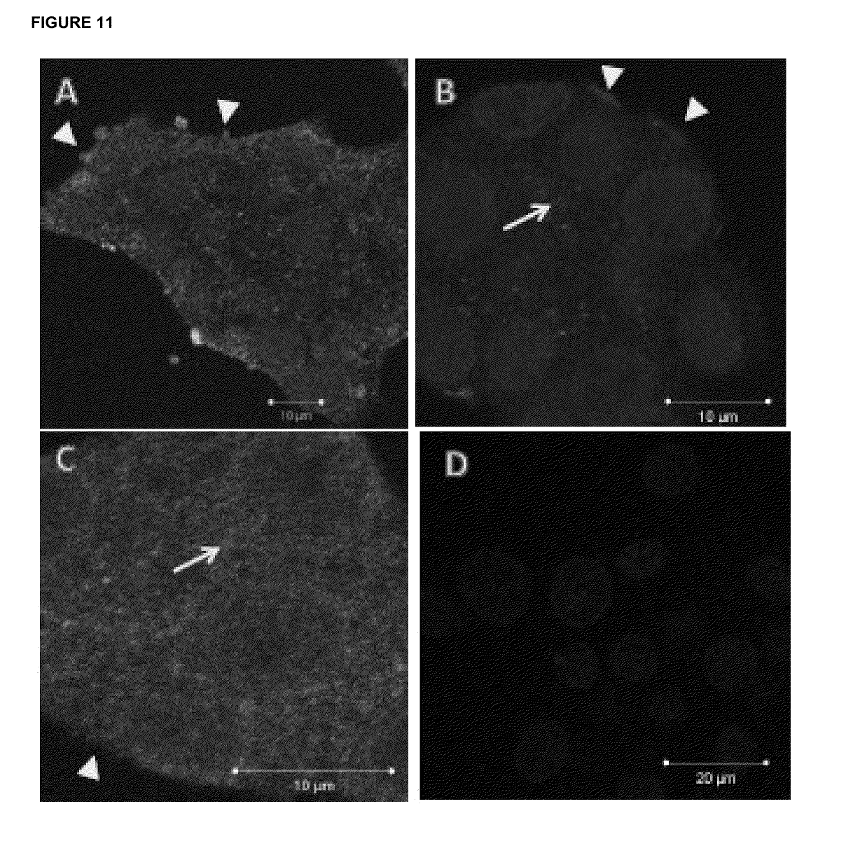

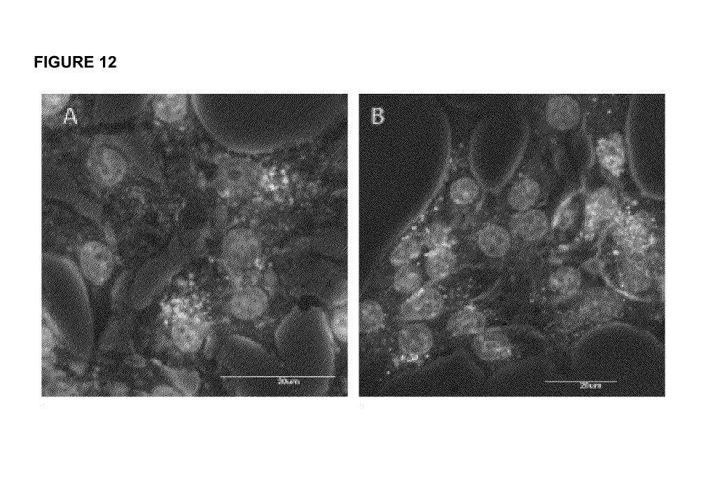

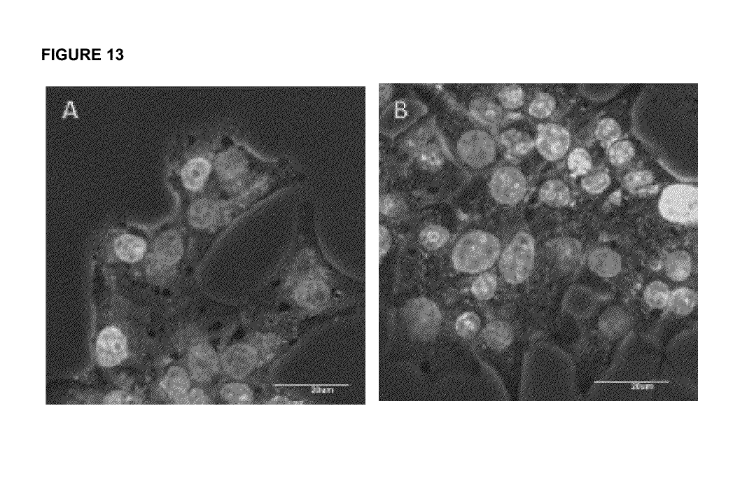

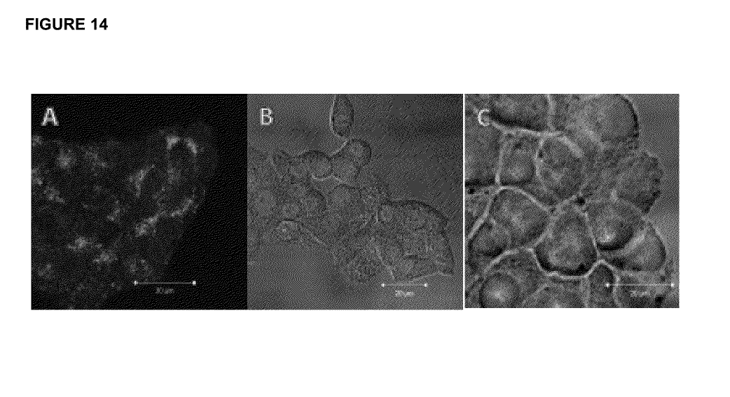

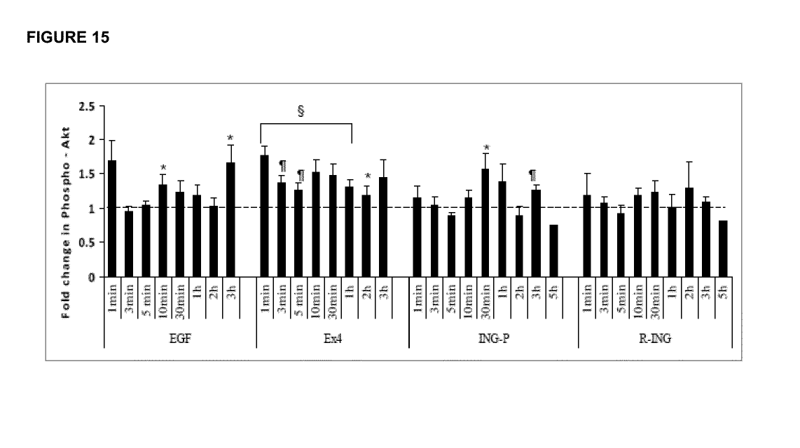

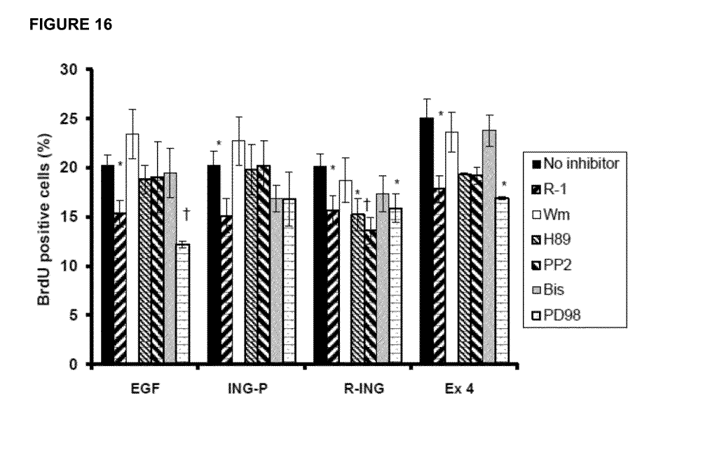

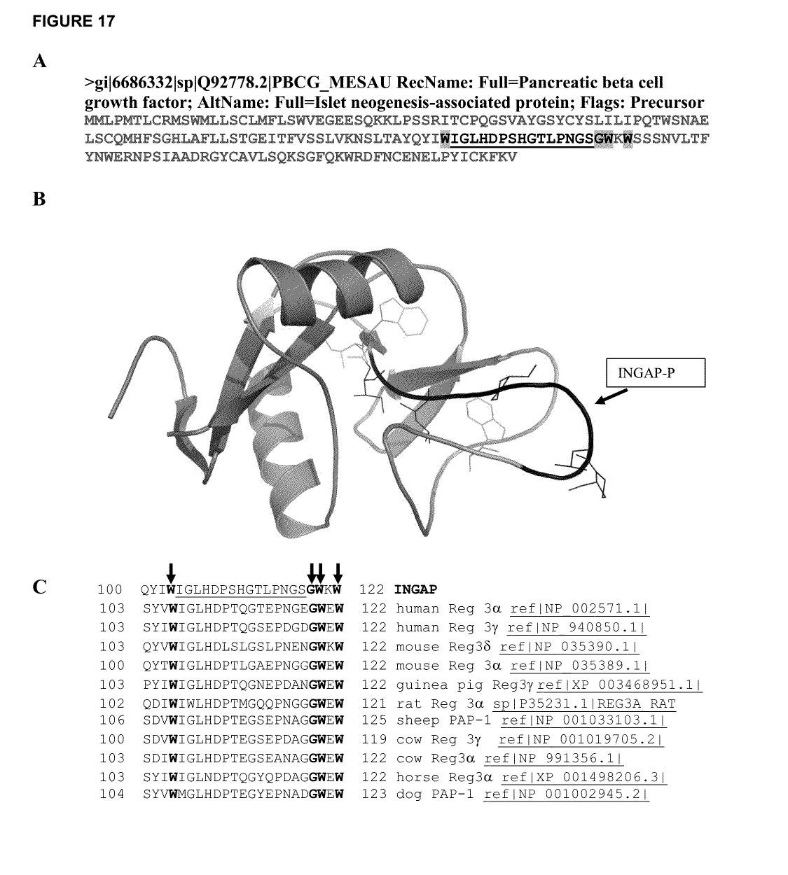

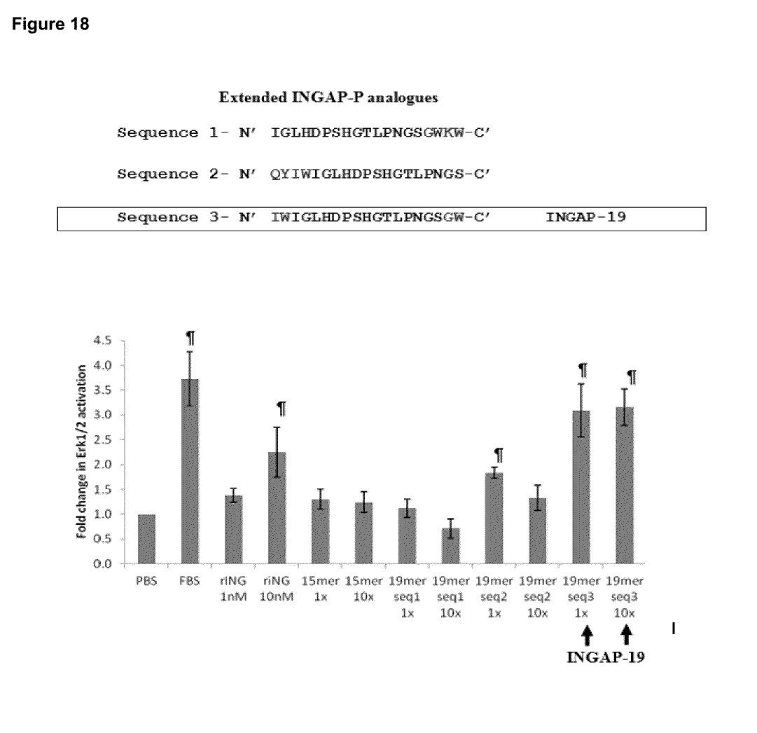

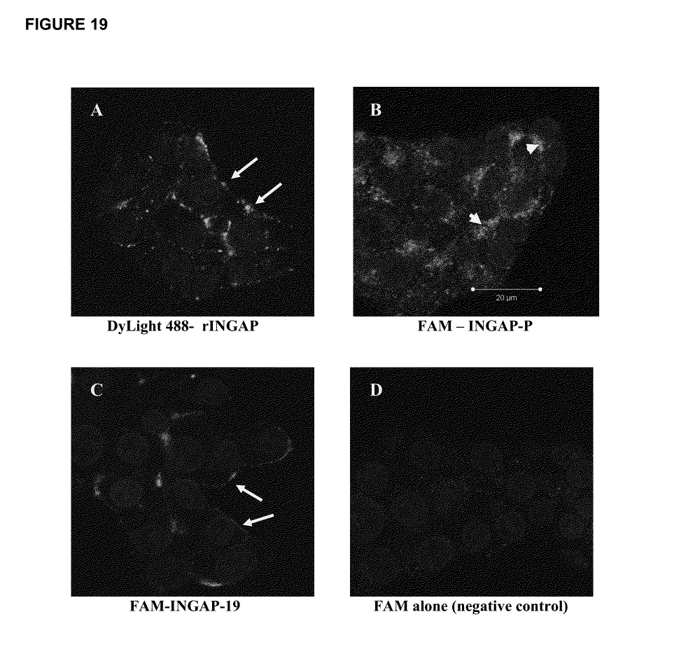

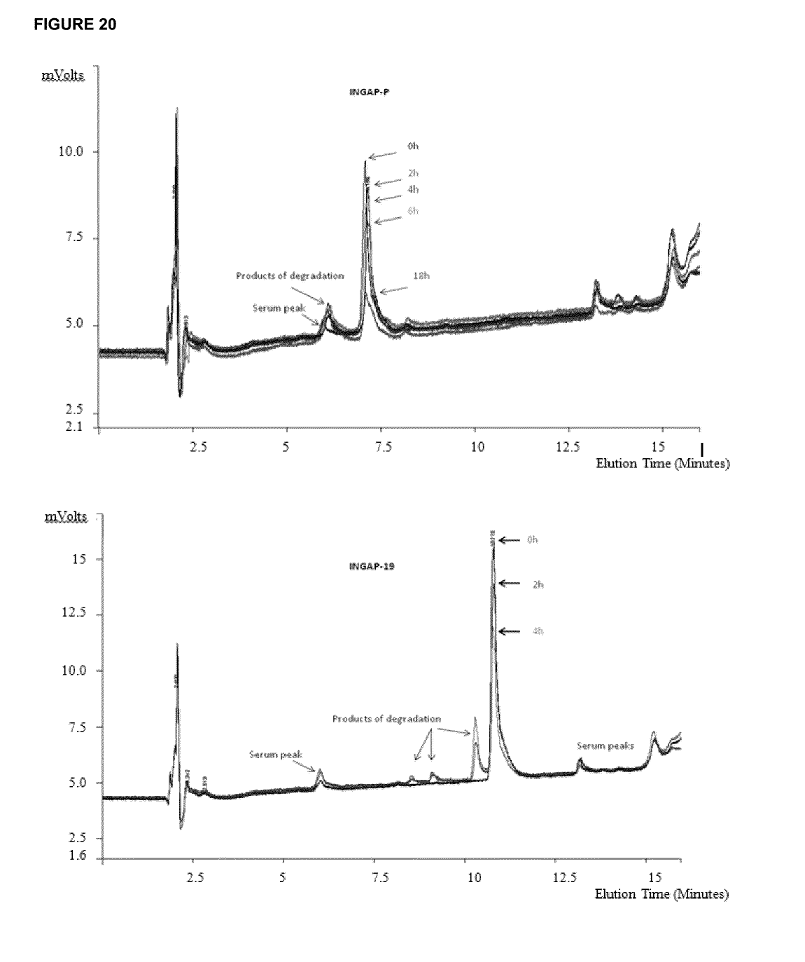

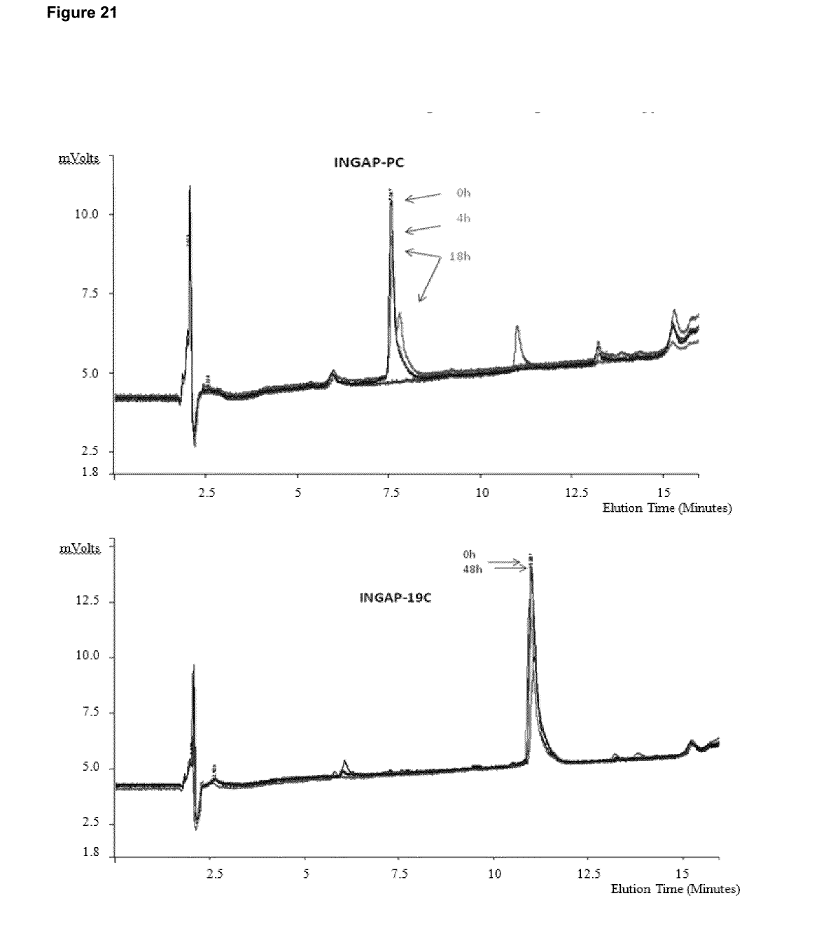

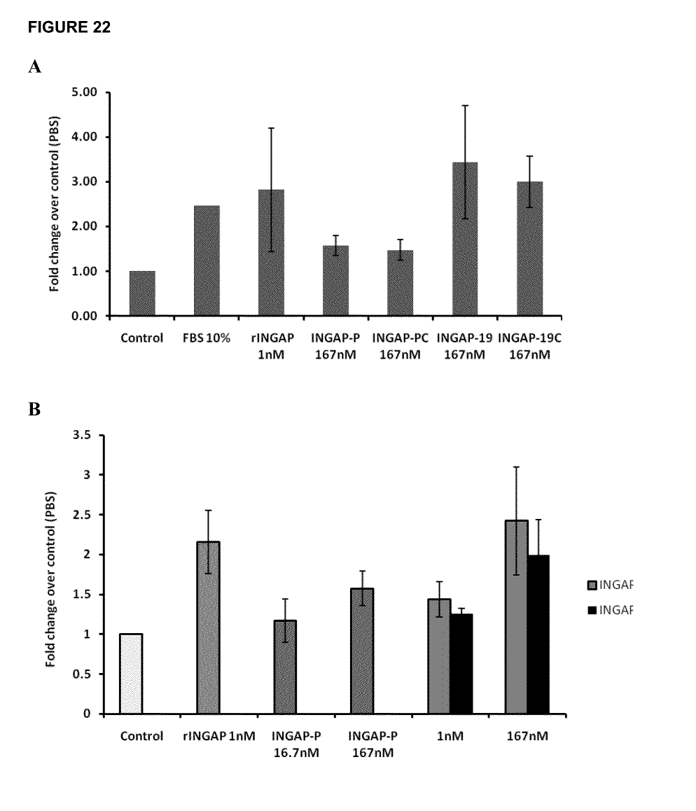

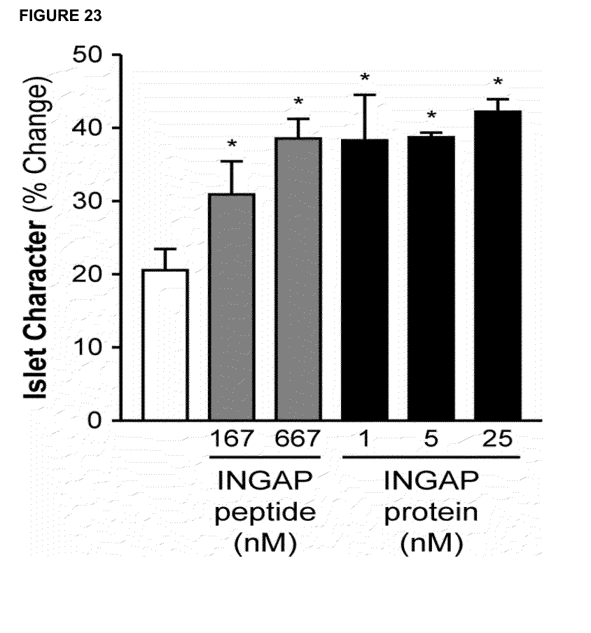

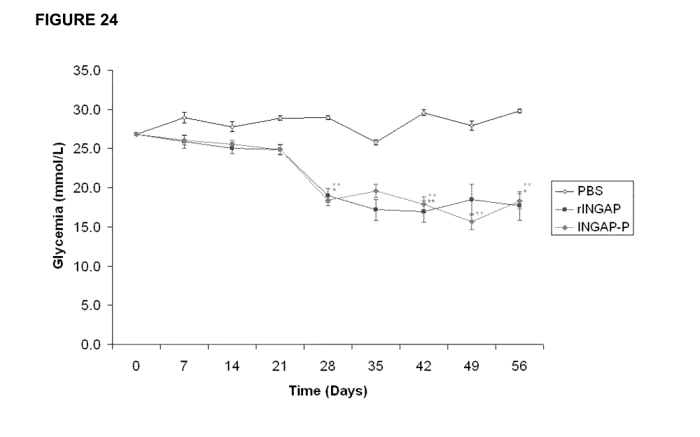

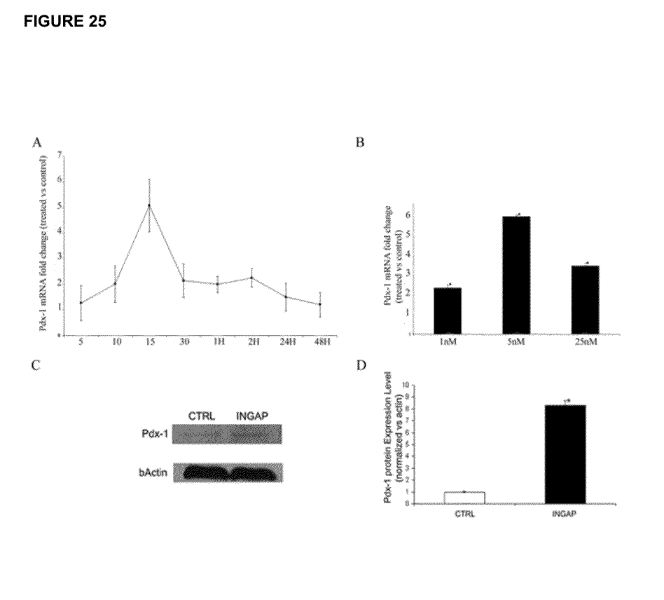

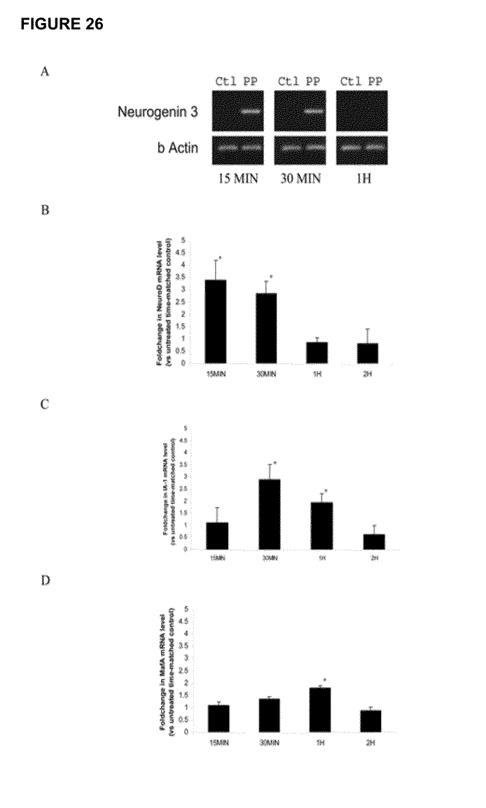

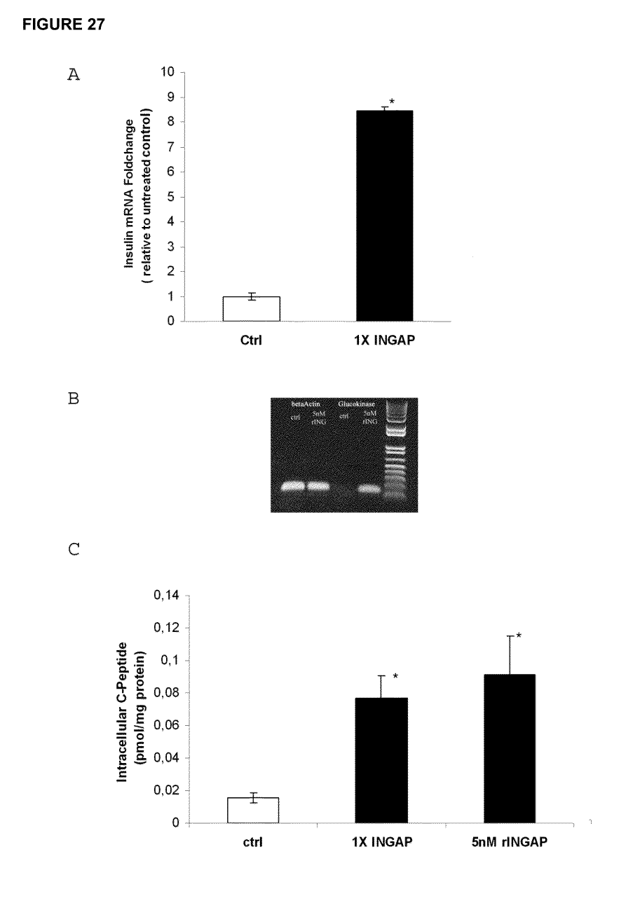

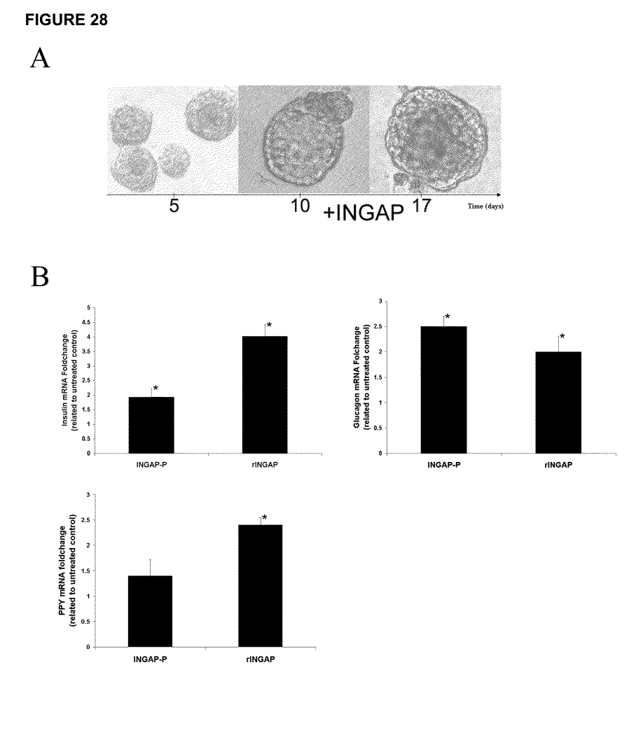

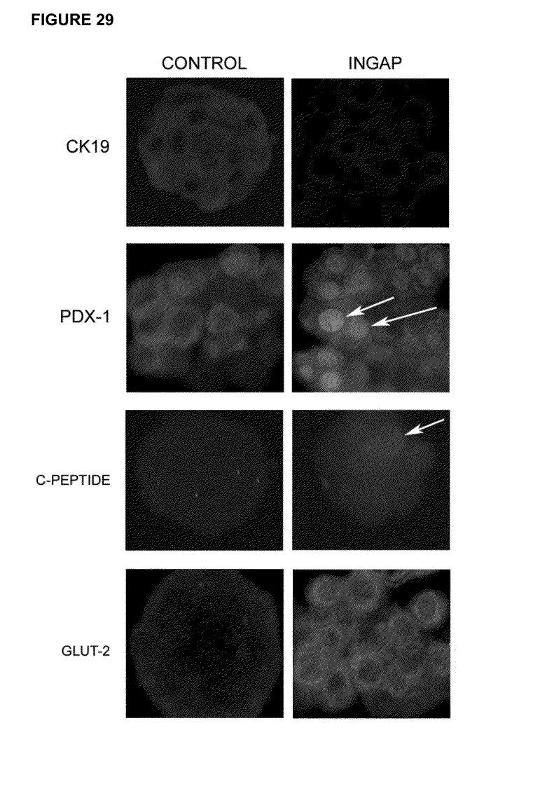

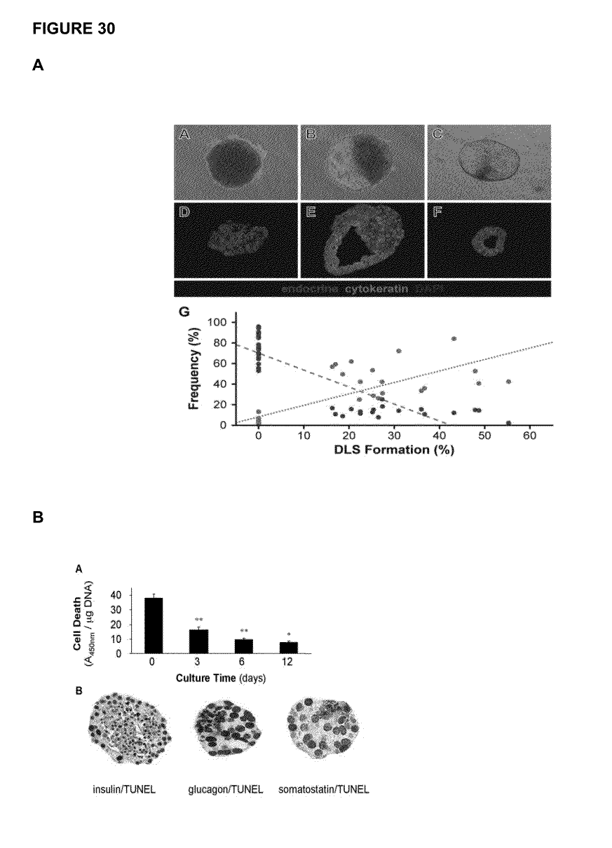

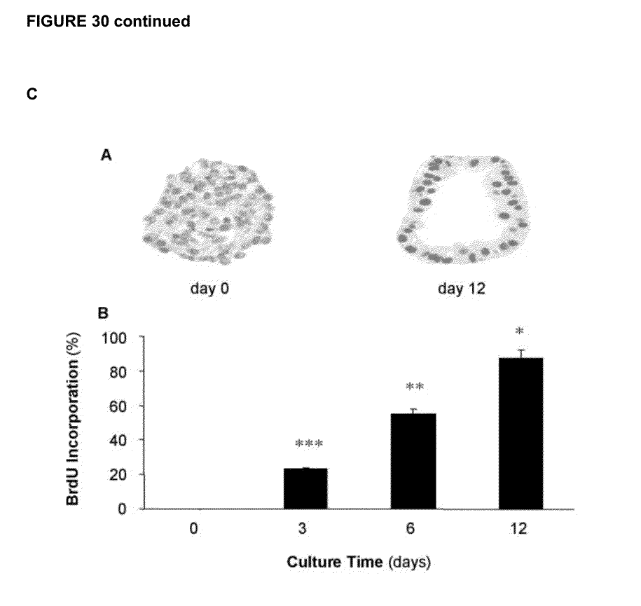

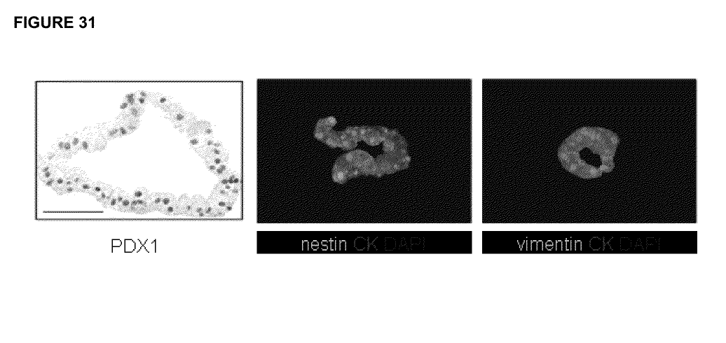

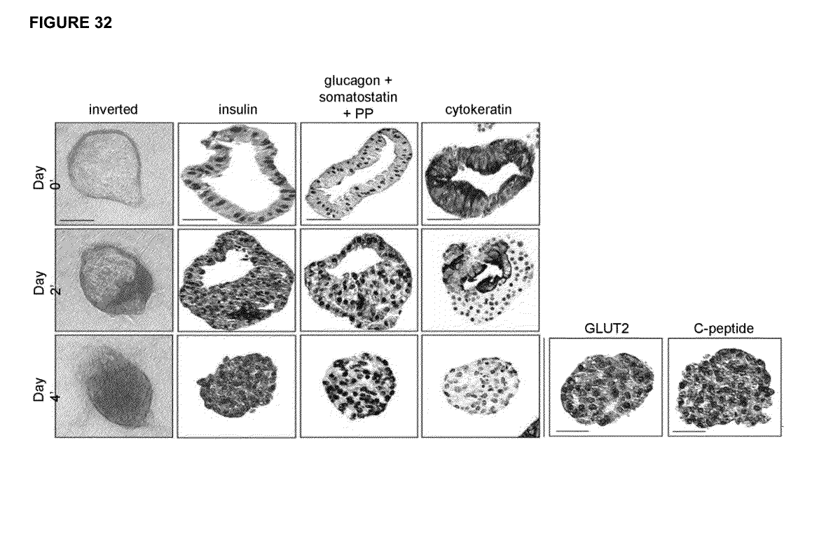

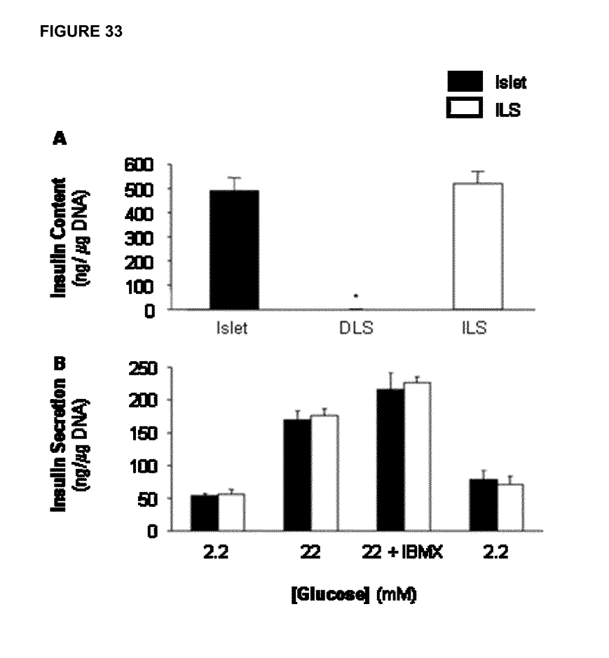

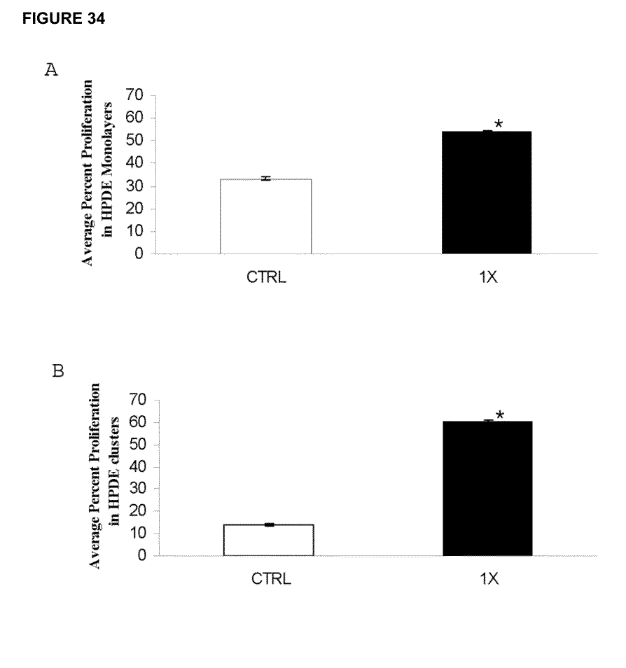

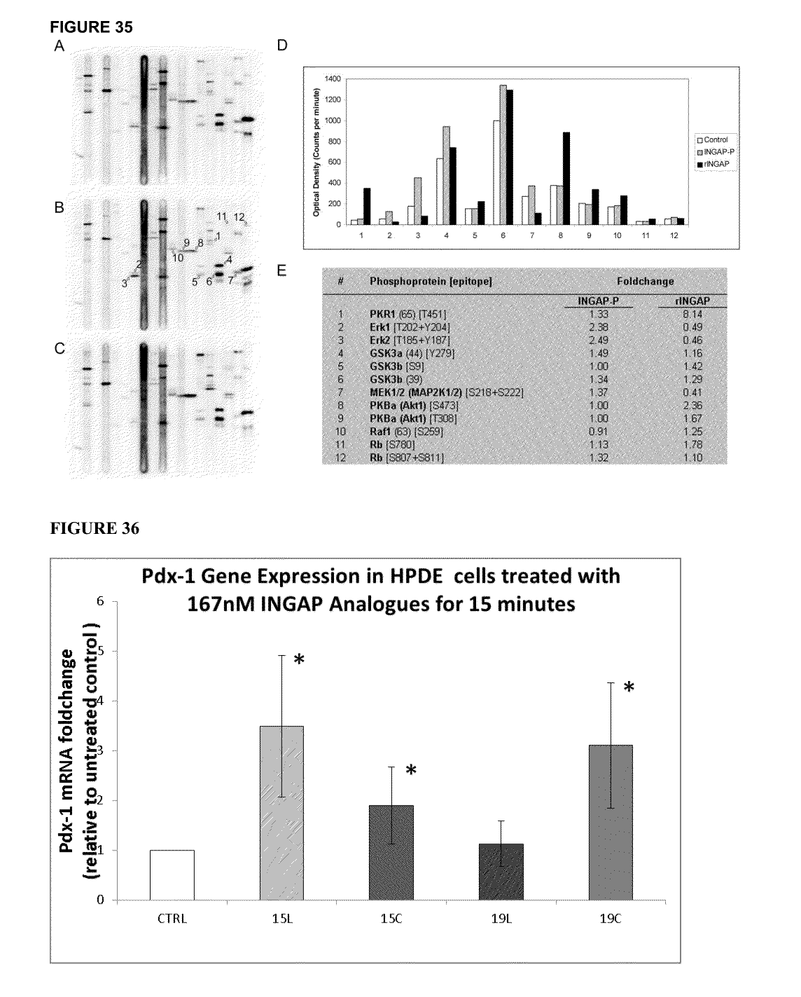

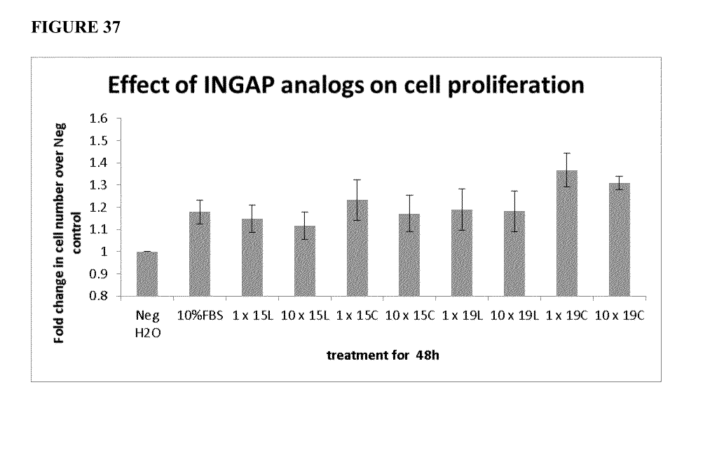

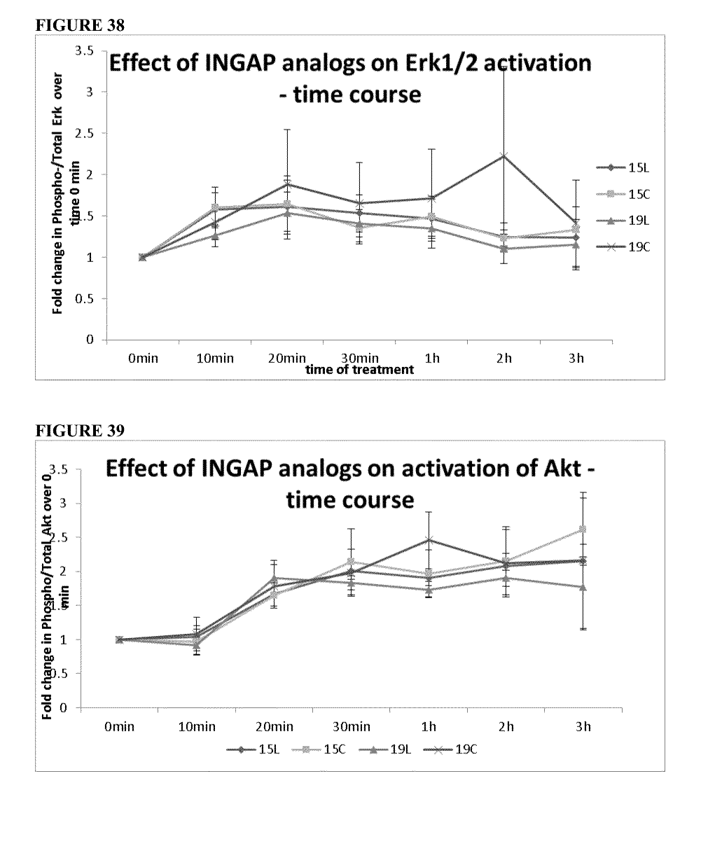

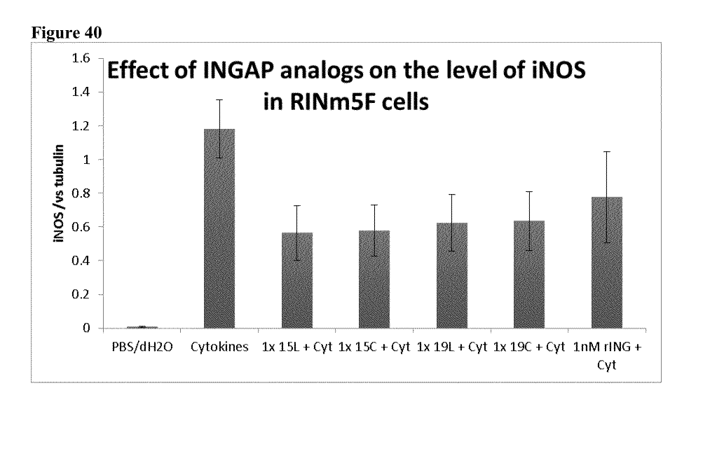

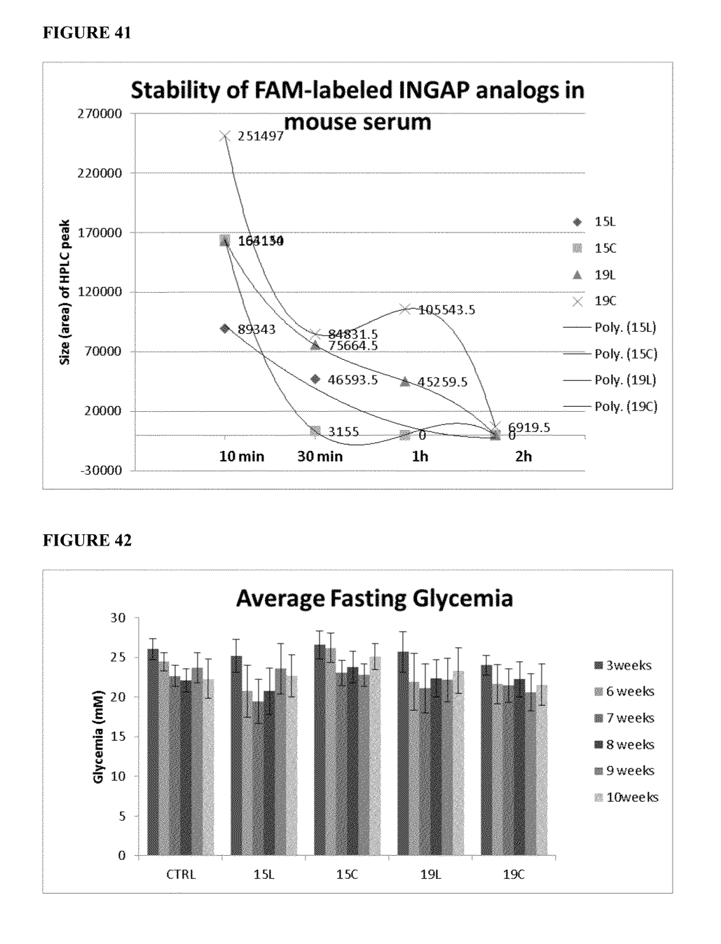

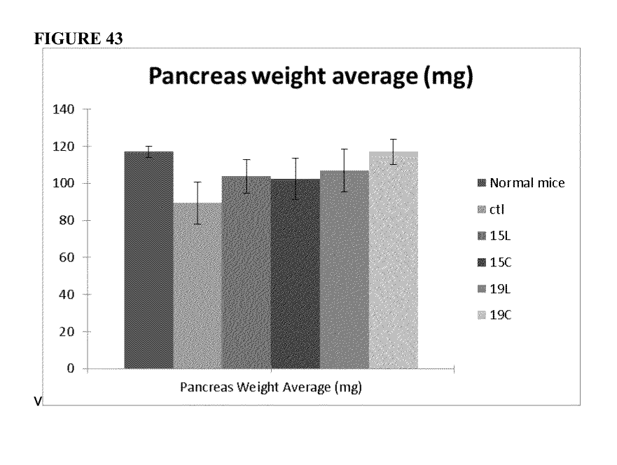

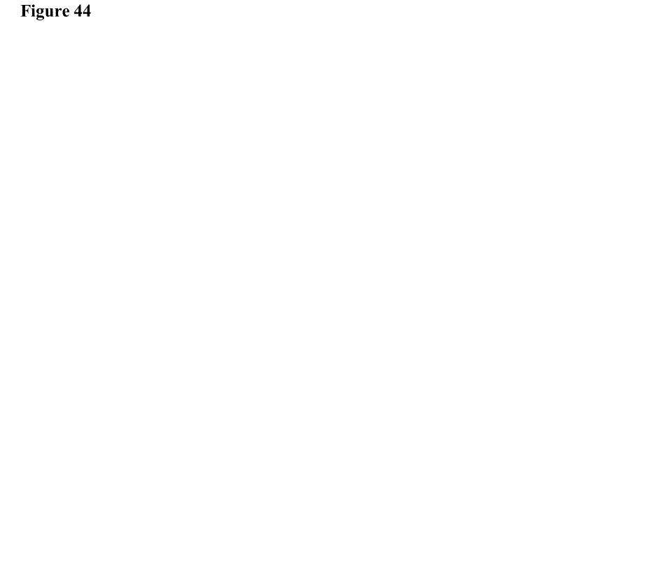

Activation of low molecular weight Ras family GTPases is the first key event in signaling through RTKs, such as EGFR. It became apparent, however, that mechanisms of MAP kinase activation by GPCRs may also include Ras activation by cross-talk between GPCRs and RTKs, e.g., transactivation of EGFR shown for several GPCR ligands, including GLP-1. In keeping with this notion, our results show a rapid Ras activation by both INGAP-P and rINGAP (FIG. 7A), consistent with a timeline of Erk½ phosphorylation (FIG. 1B,C) and c-Raf (FIG. 7B), thus implicating the Ras-Raf- MAPK pathway.

In addition to Ras activation, we observed an increase in Akt phosphorylation after 30 min of treatment with INGAP-P that was delayed relative to activation of Erk½ (FIG. 15). This suggested that activation of PI3 K/Akt signaling is parallel but not causative of Erk½ phosphorylation by INGAP-P. rINGAP also appeared to slightly elevate Akt phosphorylation, although the change was not significant. As shown in FIG. 15, strongest activation of Akt was induced by Ex-4, which was observed at early time points and which was consistent with data on GLP-1 signaling in another rat insulinoma cell line (Buteau, J., et al., 1999, Diabetologia, 42, 856-864). An early activation of Akt was also observed after EGF treatment, with an apparent secondary peak after 3 h. Taken together, these results indicate that signaling events upstream of Ras-Raf-Erk activation may vary between INGAP-P and rINGAP, and are likely different from the ones induced by Ex-4 and EGF. Of note, we did not observe significant activation of either p38 MAPK (Western blot), or PKA (ELISA), or PKC (Western blot and ELISA) by either protein or peptide (data not shown).

To investigate signaling events implicated in INGAP induced proliferation, we employed specific pharmacological inhibitors of Raf (Raf inhibitor 1), PI3 K (wortmannin), PKC (Bis), PKA (H89, PKi), Adenylate cyclase (SQ22536), Src (PP2) and EGFR (AG1478). In addition, Pertussis toxin (Ptx) was used to examine whether INGAP actions were mediated by a GPCR. Effectiveness of these inhibitors was judged by Erk½ phosphorylation after 10 min of treatment with INGAP or EGF or Ex-4, and by BrdU incorporation after 24 h.

As shown in FIG. 8A, INGAP-induced activation of Erk½ was inhibited by 40% after a 24 h exposure to Ptx , but not affected by AG1478 (FIG. 8B). This suggests that INGAP likely signals through a GPCR but that this signaling does not involve the EGF receptor, as has been previously shown for GLP-1 (Buteau, J., et al., 2003, Diabetes, 52, 124-132). Ptx also inhibited early Ras activation induced by INGAP or EGF or Ex4 (FIG. 9) which further supports the idea that INGAP signals via a GPCR-Ras pathway. Consistent with the previous implication of Ras-Raf signaling, pretreatment with Raf kinase inhibitor 1 reduced both Erk½ activation after 10 min (FIG. 8B) and BrdU incorporation after 24 h induced by all growth factors tested (FIG. 16). Interestingly, Src inhibitor PP2 inhibited both Erk½ phosphorylation (FIG. 8B) and proliferation (FIG. 16) stimulated by r-INGAP, but not by INGAP-P, which further highlights differences in signaling between protein and peptide.

Aside from expected inhibition of Erk½ and BrdU incorporation by PD98059, no other inhibitor tested (for PKC, PI3 K, or PKA) significantly reduced Erk½ phosphorylation. Except for H89 (PKA inhibitor) causing reduction in BrdU incorporation in rINGAP-treated cells after 24 h, which is discussed below, no inhibition of proliferation was observed in other groups (FIG. 16). Our earlier data clearly implicate PI3 K/Akt signaling in INGAP-P-induced islet neogenesis from human dedifferentiated duct-like structures (Jamal, A. M., et al., 2005,Cell Death Differ, 12, 702-712). This pathway appears to also mediate effects of INGAP-P on rat neonatal islets (Barbosa, H. C., et al., 2008, J Endocrinol, 199, 299-306). Moreover, PI3 K-mediated signaling appears to be the most common pathway for Reg proteins (Takasawa, S., et al., 2006, FEBS Lett, 580, 585-591; Bishnupuri, K. S., et al., 2006, Gastroenterology, 130, 137-149). In this context, our data showing no involvement of PI3 K/Akt pathway in mitogenic effects of either INGAP-P or rINGAP may seem surprising. We did, however, observe Akt phosphorylation in cells treated with INGAP-P for 30 min, which is trailing the peak in Ras-Raf-Erk activation at 1-15 min.

In contrast to the Erk½ data, an inhibitor of PKA (H89) reduced BrdU incorporation in rINGAP treated cells (FIG. 16). Given the known role of cAMP dependent PKA in GPCR signaling, and previous results implicating this pathway in a stimulatory effect of INGAP-P on neurite outgrowth in dorsal root ganglia (Tam, J., et al., 2006, Neuroreport, 17, 189-193), we performed additional experiments to examine a potential involvement of this pathway in INGAP-induced proliferation. We also tested a more specific PKA inhibitor, PKi (Murray, A. J., 2008, Sci Signal, 1, re4) and SQ22536, a specific inhibitor of adenylate cyclase, on Erk½ phosphorylation. As shown in FIG. 8A, PKi (100 nM) had no effect and SQ22536 (200 nM) not only did not reduce Erk½ phosphorylation induced by either INGAP-P or rINGAP but even slightly increased it.

Our results suggest that a cAMP-PKA pathway is not involved in INGAP signaling. In this context, if INGAP indeed signals through a GPCR, the receptor is likely coupled to a G;-protein, which has an ability to inhibit adenylate cyclase (Luttrell, L. M., 2002,Can J Physiol Pharmacol, 80, 375-382).

Taken together, results presented herein show that both INGAP-P and rINGAP stimulate proliferation in RIN-m5 F cells by activating a Ras-Raf-Erk pathway. Both INGAP-P and rINGAP likely act via a G;-protein coupled receptor(s) that does not induce activation of cAMP.

### Example 4

### Stability Testing of INGAP Peptides.

Degradation profiles of INGAP-P and INGAP-19 peptides in presence of serum were determined (FIG. 20). 50 μM peptides were incubated in RPMI-1640 medium with 25% FBS for the times indicated. Following ethanol precipitation of serum proteins, samples were analyzed by HPLC. To compare dynamics of peptide degradation HPLC profiles were superimposed as shown.

Time-course studies of in vitro incubation of INGAP-PC and INGAP-19C peptides in FBS were also performed (FIG. 21). 50 μM INGAP-PC and INGAP 19C were incubated in RPMI-1640 medium with 25% FBS for the times indicated. Following ethanol precipitation of serum proteins, samples were analyzed by HPLC. To compare dynamics of peptide degradation, HPLC profiles were superimposed as shown. It can be seen that no degradation was observed for INGAP-19C for 48 h in presence of serum (FIG. 21B). Of the INGAP peptides tested, INGAP-19C showed the highest stability.

### EXPERIMENTAL PROCEDURES

### Recombinant INGAP Protein and INGAP Peptides

A 15-amino acid fragment of INGAP protein (amino acids 104-118) and a 19-amino acid fragment of INGAP protein (amino acids 102-120) were synthesized and HPLC-purified at the Sheldon Biotechnology Centre (McGill University, Montreal). A full-length recombinant INGAP (r-INGAP) containing C-terminal 6-His tag (MW 17.6 kDa) was cloned from hamster pancreatic tissue by directional cloning of a PCR product generated with Superscript III RT and Platinum™ Pfx DNA Polymerase (Invitrogen) into the pcDNA3.1D/V5-His-TOPO™ expression vector (Invitrogen). This construct was used for re-cloning into a lentiviral vector and expressed in H293 cells (as described in Assouline-Thomas, B., et al., 2010, Protein Expr Purif, 69: 1-8). Purification of r-INGAP was carried out using Cobalt resin (BD TALON™, BD Biosciences, or Fractogel EMD Chelate(M), Merck) as described (Assouline-Thomas, B., et al., 2010, Protein Expr Purif, 69: 1-8).

Cell culture

RIN-m5 F cells (passage 18) were purchased from ATCC and maintained at 37° C./5% CO₂ in RPMI-1640 medium (Invitrogen) containing 25 mM glucose, 10% FBS (Montreal Biotech), and antibiotics/antimycotics (Invitrogen). Experiments were carried out on cells from passages 25-31.Cells were plated in 60 mm tissue culture dishes (1×10⁶ cells per dish) and allowed to grow for 24-48 h, followed by serum withdrawal for 24 h prior to treatment with INGAP proteins or peptides. INGAP-P (15-mer peptide), INGAP-P2 (19-mer peptide), rINGAP, and EGF (10 ng/ml, Sigma) were administered in serum-free medium for the times indicated.

### Assessment of Cell Proliferation by BrdU Immunostaining

Cells plated in 8-well or 4-well chamber slides (5×10⁴ or 1×10⁵ cells per well) were treated for 24 h with INGAP, EGF or Ex 4, as described above, and 50 μM BrdU was added during the last 3 hours of treatment. Cells were washed with PBS and fixed in Methanol for 10 min at −20° C. Immunostaining for BrdU was carried out using mouse anti- BrdU antibody (Roche) following the manufacturer's protocol. This was followed by detection with secondary, HRP-conjugated antibody (broad spectrum, Histostain™-Plus) and AEC chromogen (both from Zymed Laboratories). Slides were counterstained with hematoxylin. BrdU-positive and negative nuclei were counted (total 200 per well) and the percentage of BrdU-positive nuclei was calculated (FIG. 36).

### Western Blot Analysis

Following treatments, cells were placed on ice, washed with PBS and solubilized in lysis buffer (Cell Signaling, Inc., Beverly, Mass.), containing 2.5 mM Na₄ P₂ O₇ , 1 mM Na₃VO₄ and Complete protease inhibitor cocktail tablet (Roche). Equal amounts of protein (20-50 m, measured with DC Protein assay (Bio-Rad)) were resolved by 10% SDS-PAGE, followed by transfer onto Nitrocellulose membrane (Bio-Rad) at 250 mA for 90 min and analyzed with different antibodies. Anti-Erk½ (MAPK 44/42) and anti-phospho Erk½ (Thr202/ Tyr204) rabbit polyclonal antibodies were purchased from Cell Signaling. Following primary antibody incubation, blots were washed and then incubated in a secondary, anti-mouse or anti-rabbit HRP-conjugated antibody (Cell Signaling), and washed and developed using the ECL system (GE Healthcare). To analyze expression of several proteins on the same blot, membranes were first incubated with phospho-antibodies followed by stripping (0.2 M Glycine, 0.1% SDS, 0.05% Tween20, pH2.2) prior to probing with corresponding non-phospho primary antibodies.

Visualization of fluorescent rINGAP, INGAP¹⁰²⁻¹²⁰ and INGAP¹⁰⁴⁻¹¹⁸

100 μg of rINGAP were labeled with DyLight-488 or DyLight-594 (ThermoScientific) as specified in the instructions. INGAP¹⁰²⁻¹²⁰ and INGAP¹⁰⁴⁻¹¹⁸ were labeled with either 5-FAM or FITC during synthesis at the Sheldon Biotechnology Centre (McGill University, Montreal) or Canpeptide (Pointe Claire, Quebec). Fluorescent rINGAP(50 nM) or INGAP¹⁰²⁻¹²⁰ and INGAP¹⁰⁴⁻¹¹⁸ (8.35-16.7 μM were added to RIN-m5F cells grown in glass chamber slides (Beckton-Dickinson or Lab-Tek), for various intervals followed by washing with PBS and fixation in 4% paraformaldehyde. Slides were mounted using VectaShield medium (Vector) or Prolong Gold (Invitrogen) with DAPI for counterstaining of nuclei and examined under confocal microscope Zeiss LSM 510 or Olympus FV10i. For live confocal imaging cells were grown in Nunc™ chambered coverglass slides (ThermoScientific). Nuclei were stained with 0.01% DAPI prior to incubation with INGAP followed by washing. Live imaging was carried out at 37° C. and 5% CO₂.

### Statistical Analysis

Experiments were repeated at least three times. Results are expressed as means±SEM. Statistical analysis was performed with unpaired Student's t-test. A p-value of <0.05 was considered significant.

### Example 5

### Comparison of 15 L INGAP and 15C INGAP

Procedure: Cell plating and treating:

Aspirate all FBS-containing medium from a plate of RINm5 F cells and pipette 10 mL of PBS into the plate in order to wash off the medium. Aspirate the PBS and pipette 600 μL of trypsin into the plate. Tilt the plate to ensure that the trypsin covers everything. Add 8 mL of FBS-containing medium to the plate and then collect the mixture of medium and cells into a 15 mL tube. Count the cells using a 1:1 dilution of cells and trypan blue using a hemocytometer under a microscope in order to determine the cell density. Calculate the amount of cells needed in each plate in order to get the wanted cell density of 5×10⁵ cells/plate and plate the cells into 16-35 mm plates. Incubate cells for 3 days at 37°, 5% CO₂. Switch the medium from serum-containing medium to FBS-free medium in order to serum starve the cells for 24 hours before treatment. Treat the 16 plates:
a. 4 plates with 15C INGAP
b. 4 plates with 15L INGAP
c. 4 plates with FBS
d. 4 plates with H₂O

Incubate the plates for 48 hours and collect the cells from each plate through trypsinization and place each sample in its own Eppendorf tube.

### Cell Viability Assays

Cell Viability Assays were conducted by mixing 5 μL of cell from 1 sample with 10 mL of the cell counting machine's salt solution in a vial Place the vial inside the probe. The machine will count the number of live cells. Repeat the cell count with all of the other cell samples, doing two separate counts per sample. Conduct a Bradford Assay as shown below in order to normalize the cell counts to total protein. The results are shown in Tables 1A-C below.

TABLE 1A
Comparison of the Effect of 15L INGAP and 15C INGAP on Cell
Proliferation Through a Cell Viability Assay:

| | Average | | |
|---|---|---|---|
| | Count/Protein | Standard | Standard |
| Condition | Concentration | Deviation | Error of Mean |
| H2O | 146583.33 | 16219.70 | 8109.85 |
| FBS | 485041.67 | 15393.83 | 7696.91 |
| 15L INGAP | 487593.75 | 24409.54 | 12204.77 |
| 15C INGAP | 656227.27 | 19065.57 | 9532.78 |

TABLE 1B
Cell Viability One-Way Analysis of Variance (ANOVA)

| Source of | | | | | |
|---|---|---|---|---|---|
| Variation | SS | dF | MS | F | P-Value |
| Between | 5.48 × 10¹¹ | 3 | 1.83 × 10¹¹ | 281.57 | 2.2 × 10−11 |
| Groups | | | | | |
| Within | 7.79 × 10⁹ | 12 | 6.49 × 10⁸ | | |
| Groups | | | | | |
| Total | 5.56 × 10¹¹ | 15 | | | |

TABLE 1C
Cell Viability Post-Hoc Independent T-tests:

| | 15C to 15L | 15C to H₂O | 15L to H₂O |
|---|---|---|---|
| P Value | 0.00032 | 2.02 × 10−8 | 4.48 × 10−6 |
| T Value | 5.16 | 18.32 | 6.67 |
| Degrees of Freedom | 3 | 3 | 3 |
| (df) | | | |

### Bradford Assay

A Bradford assay was conducted to normalize the cell counts to total protein. The samples were centrifuged and the medium aspirated and replaced with 1 ml of PBS per tube and centrifuged again. The PBS was aspirated and replaced with 200 μL of RIPA lysis buffer. The centrifugation was repeated in a 4° C. room. 5 μL of 8 different “standard” concentrations of BSA dissolved in RIPA lysis buffer was pipetted, into the first three columns of a 96 well plate (triplicates of each concentration), the final concentration being a blank, only containing lysis buffer. These will be used later for calculation purposes. A duplicate of each protein sample was centriguged. About 2 mL of Bradford reagent was prepared by mixing together 2 mL of solution A from a Bio-Rad protein assay kit and 40 μL of solution S, also from the kit. 254, of the Bradford reagent was placed via pipette into each well 200 μL of solution B from the kit was placed into each well. The plates were shaken for 10 minutes in order to mix the solutions together (make sure there are no bubbles) and placed in a reader to calculate the absorbance of the each sample. The resulting values were entered into a spreadsheet. A standard curve was created using the already known concentration of the standards (x-values) and their absorbance values (y-values). The formula of the line of best fit was used to algebraically calculate the concentration of the samples by plugging in their absorbance values

### Assessment of Cell Proliferation by Brdu Immunostaining

Cells were plated in three 8-well chamber slides (1×10⁵ cells per well) were treated with 15C INGAP 15 L INGAP, water or FBS and incubated overnight, and 50 82 M BrdU was added during the last 3 hours of treatment. Cells were washed with PBS and fixed in Methanol for 10 min at −20° C. Immunostaining for BrdU was carried out using mouse anti-BrdU antibody (Roche) following the manufacturer's protocol. This was followed by detection with secondary, HRP-conjugated antibody (broad spectrum, Histostain™-Plus) and AEC chromogen (both from Zymed Laboratories). Slides were counterstained with hematoxylin. BrdU-positive and negative nuclei were counted (total 200 per well) and the percentage of BrdU-positive nuclei was calculated. The results are shown in Tables 2A-C below.

TABLE 2A
Comparison of the Effect of 15L INGAP and 15C INGAP on Cell
Proliferation Through BrdU Staining:

| | Average | | |
|---|---|---|---|
| | Percentage of | | |
| | BrdU Stained | Standard | Standard |
| Condition | Cells (%) | Deviation | Error of Mean |
| H2O | 6.52 | 0.71 | 0.35 |
| FBS | 10.99 | 2.41 | 1.20 |
| 15L INGAP | 9.69 | 1.04 | 0.52 |
| 15C INGAP | 16.19 | 0.96 | 0.48 |

TABLE 2B
BrdU One-Way (ANOVA):

| Source of | | | | | |
|---|---|---|---|---|---|
| Variation | SS | dF | MS | F | P-Value |
| Between | 194.17 | 3 | 64.72 | 31.18 | 6.02 × 10⁻¹¹ |
| Groups | | | | | |
| Within | 24.91 | 12 | 2.08 | | |
| Groups | | | | | |
| Total | 219.08 | 15 | | | |

TABLE 2C
BrdU Post-Hoc Independent T-tests:

| | 15C to 15L | 15C to H₂O | 15L to H₂O |
|---|---|---|---|
| P Value | 9.39 × 10−5 | 3.54 × 10−6 | 0.0024 |
| T Value | 12.99 | 20.34 | 6.40 |
| Degrees of Freedom | 3 | 3 | 3 |
| (df) | | | |

### Western Blot Analysis

### Western Blot for Phospho-ERK: Foll

Following the cell plating and treating procedure 16 plates were treated with different treatments and for different amounts of time:
a. 2 plates of 10×15C INGAP left for 5 minutes
b. 2 plates of 10×15C INGAP left for 10 minutes
c. 2 plates of 10×15C INGAP left for 30 minutes
d. 2 plates of 10×15C INGAP left for 60 minutes
e. 2 plates of 10×15 L INGAP left for 5 minutes
f. 2 plates of 10×15 L INGAP left for 10 minutes
g. 2 plates of 10×15 L INGAP left for 30 minutes
h. 2 plates of 10×15 L INGAP left for 60 minutes

Following incubation, the plates were washed 2 times with cold PBS to be sure that all of the INGAP treatment is removed from the plates. The cells were lysed by adding 200 μL of RIPA lysis buffer to each plate and placed on a shaker for approximately 20 minutes to ensure complete lysis. Protein samples were collected into Eppendorf tubes using a pipette and centrifuged at 16.1×1000 rpm for 20 minutes in a 4° C. room. The resulting supernatant was transferred into fresh tubes and a Bradford Assay conducted to determine the concentration of protein in each sample. Solve for the amount of protein needed when loading 15 μg total into each well of the gel. The samples were combined with loading buffer, heated at 100° C. for 5 minutes and placed into 2-10% acrylamide gels with 10 wells along with a ladder and run at 200 V for 50 minutes or until the blue protein front runs out the bottom. A double transfer sandwich was used to transfer the proteins from the gel onto a positively charged nitrocellulose membrane by running the transfer at 0.3A for 1 hour. This transfers the already-run proteins from the gel substance onto the membranes. The membrane was placed a clear container and the non-specific proteins blocked by soaking in 5% BSA TBST buffer for 1 hour, letting it sit on a shaker during this time. T

The blocking solution was removed and the membrane allowed to remain in a primary antibody solution (10 μL of rabbit anti-phospho-ERK antibody, which binds to phosphorylated ERK in 10 mL of 5% BSA TBST) and left it to shake overnight in a room at a temperature of 4° C. The primary antibody was removed and the membrane washed with ˜10 mL of 1 ×TBST buffer, letting it sit on the shaker for 10 minutes. The wash was repeated 3 times. The membrane was then placed in a secondary antibody solution (10 μL of goat anti-rabbit antibody, which binds to the primary antibody, in 10 mL of 5% BSA TBST, giving it a dilution factor of 1:1000) for 1 hour on the shaker at room temperature. This causes the protein to give off chemiluminescence, therefore it can be visualized using a machine. The membrane was treated with the secondary antibody twice. The membranes was covered with 1 mL of ECL solution for 5 minutes and placed into the chemi-doc machine which can visualize the phosphorylated ERK. The computer program Image Lab was used to quantify the bands of phospho-ERK on the gel.

Following quantification of phosphor-ERK, the membrane was washed with TBST to remove the ECL solution and the antibody process repeated with rabbit anti-ERK as the primary antibody instead of rabbit anti-phospho-ERK with the goal of quantifying total ERK instead of phospho-ERK. a total of 3 Western Blot trials. The results are shown in Tables 3A-C.

TABLE 3A
Comparison of the Effects of 15L INGAP and 15C INGAP
on ERK V2 Phosphorylation Through a Western Blot:

| | | | | | | Standard |
|---|---|---|---|---|---|---|
| | | | | | Standard | Error of |
| Condition | Exp 1 | Exp 2 | Exp 3 | Average | Dev | Mean |
| 15L-5 min | 0.84 | 1.71 | 0.92 | 1.16 | 0.48 | 0.28 |
| 15L-10 min | 1.02 | 1.48 | 1.41 | 1.30 | 0.25 | 0.14 |
| 15L-30 min | 0.73 | 1.21 | 1.19 | 1.05 | 0.27 | 0.16 |
| 15L-60 min | 0.70 | 1.29 | 1.47 | 1.15 | 0.40 | 0.23 |
| 15C-5 min | 0.65 | 0.92 | 1.47 | 1.01 | 0.42 | 0.24 |
| 15C-10 min | 1.29 | 2.24 | 1.86 | 1.80 | 0.48 | 0.28 |
| 15C-30 min | 2.09 | 3.68 | 2.79 | 2.85 | 0.80 | 0.46 |
| 15C-60 min | 1.70 | 1.70 | 1.21 | 1.28 | 0.39 | 0.23 |

TABLE 3B
Western Blot Two-Way ANOVA:

| | Sum of | | Mean | | | |
|---|---|---|---|---|---|---|
| 12 | Squares | DF | Square | F score | P< | Final DF |
| Treatment | 1.95 | 1 | | 9.06 | 0.01 | (1,16) |
| Time | 2.68 | 3 | 0.89 | 4.15 | 0.05 | (3,16) |
| Within | 3.44 | 16 | 1.12 | | | |
| Both | 3.36 | 3 | 0.22 | 5.22 | 0.05 | (3,16) |
| Total | 0.65 | 0.92 | 1.47 | 1.01 | 0.42 | 0.24 |

TABLE 3C
Western Blot Post-Hoc Independent T-tests:

| | 5 min 15C to | 10 min 15C to | 30 min 15C to | 60 min 15C to | 30 min 15C to |
|---|---|---|---|---|---|
| | 5 min 15L | 10 min 15L | 30 min 15L | 60 min 15L | 10 min 15L |
| P Value | 0.71 | 0.19 | 0.02 | 0.71 | 0.03 |
| T Value | | | 3.23 | | 2.79 |
| Degrees of | 2 | 2 | 2 | 2 | 2 |
| Freedom | | | | | |
| (df) | | | | | |

### Example 5

### Comparison of Stability and Bioactivity of the INGAP-Peptide Derived Analogs

The objective of this study is to expedite development of more potent and stable analogs of INGAP-peptide, as a next generation of islet regenerating agents. INGAP-peptide contains 15 amino acids (aa), which appear to represent a bio-active segment of the full-length INGAP protein. In the preliminary phase of this research, we extended the 15-mer INGAP-peptide by adding two amino acids on each end, as per protein sequence, resulting in a 19-mer, which we also cyclisized by adding Cysteines on each terminus (total 21 aa). Similarly, we produced a cyclic version of 15-mer INGAP (total 17 aa).

The resulting four analogs were denoted as 15 L (original 15-mer peptide), 15C (cyclic version of 15 L); 19L (linear 19-mer) and 19C (cyclic 19-mer).

Although 15C was not included in the proposal of the awarded CIHR POP grant, which was focused on evaluation of the 19-mer analogs, we used the 15C peptide along in all experiments to obtain as much information as possible on the effect of cyclization and extension on INGAP's biological functions

According to the research design, the experiments with the INGAP analogs were carried out both in vitro (Aim1) and in vivo (Aim2).

Aim 1: Biological Effects of INGAP Analogs In Vitro

In this part of the study we used two cell lines previously shown to be responsive to INGAP-peptide and INGAP-protein:
1. HPDE cells (human pancreatic ductal epithelial cells) were used for a differentiation assay
2. RIN-m5F (rat insulinoma cells, a surrogate model of beta cells) were used for the analysis of proliferation and the associated signaling events, as well as in the model of cytokine-induced apoptosis (rescue from apoptosis)

HPDE Cells—Differentiation Assay

Pdx-1 (Pancreatic and duodenal homeobox 1) is expressed in the pancreatic duct epithelium cells during development, and seems to be a prerequisite for their differentiation into acini, ducts, and endocrine cells in the mature pancreas We investigated Pdx-1 gene expression in INGAP-treated HPDE cells.

There is a basal low level of Pdx-1 in adult pancreatic ductal cells. We have previously shown that the level of Pdx-1 mRNA increases after 15 minutes of treatment with 167 nM INGAP-P, this effect is rapidly followed by the induction of a cascade of transcription factors that lead to insulin expression, and therefore reflect a transdifferentiation of adult ductal cells towards a beta-like phenotype (effect described for INGAP-P in “Islet Neogenesis Associated Protein (INGAP) Induces the Differentiation of an Adult Human Pancreatic Ductal Cell line into Insulin-expressing Cells through Stepwise Activation of Key Transcription Factors for Embryonic Beta Cell Development.” Assouline et al, manuscript submitted to Differentiation).

Here we have compared the effect of the 4 different analogues on the expression of Pdx-1 by qPCR.

Referring to FIG. 36, INGAP induces Pdx-1 expression in human adult ductal cells. Pdx-1 mRNA expression in HPDE cells treated with 167 nM INGAP-P (15 L), −15C, 19 L or 19C is expressed as a fold-change of the time-matched untreated control. Data is presented as mean±SEM, *p<0.05 (t-test), n=4 independent measurements.

Our data show that all 4 peptides do induce an increase of Pdx-1 expression after 15 minutes. The two more potent molecules at the concentration of 167 nM tested in this experiment are INGAP-P (15 L) and INGAP-19C which induce an increase of 3.49±0.70 fold and 3.10±0.48 fold respectively. INGAP-15C also induces a smaller increase of the level of Pdx-1 mRNA: 1.89±0.33 fold. INGAP-19 L induces an increase as well, but not significant at this time point.

In conclusion, all four peptides do increase Pdx-1 mRNA levels, what suggest at the light of our previous data with INGAP-P, that they all can instruct the cells to undergo the first steps of endocrine differentiation.

Proliferation Assay (RINm5F Cells

In the previous study, we showed that INGAP-peptide (15 L) dose-dependently increased proliferation of RIN-m5 F (by 60% after 24 h, BrdU assay) and activated Erk½ by phosphorylation on Thr202/ Tyr204that peaked at 10 min of treatment (Petropavlovskaia M et al., 2012, Am J Physiol Endocrinol Metab). The effect was observed at concentration of 167 nM and higher, whereas lower concentrations, e.g 16.7 nM, had no effect.

In the current study we assessed the proliferative effects of all four analogs on RIN cells by cell counting after 48 h and by measuring Erk phosphorylation in a time-course experiments. The analogs were used at 167 nM (1×) and 1.67 mM (10 ×) concentrations.

The results of the 48 h treatment by cell counting (FIG. 37) indicated that all peptides were able to increase proliferation of RIN cells, similar to the positive control (10% FBS) with 19C being the most effective, especially at 1× concentration.

Referring to FIG. 37, RINm5 F Cells plated in 6-well plates at 5×10⁵ in 10% FBS RPMI medium for 48 h were serum starved overnight and treated with INGAP analogs (triplicate wells) in serum-free medium for 48 h, trypsinized and counted in Coulter counter. The data shown are average of three experiments.

Signaling events leading to the increase in proliferation, i.e., Erk phosphorylation, occur early after addition of INGAP. We compared the dynamics of Erk½ activation by the analogs in a time course experiments lasting up to 3 h (FIG. 38). The analogs were used at 10× (1.67 mM) concentration. 25 mg of total protein was used for Western Blot analysis for both phosphorylated and total Erk½ and the ratio of phospho-/total was used as a measure of Erk½ activation. The values were normalized by the time 0 min thus showing Fold change in Erk½ activation over time. Shown are the results of four experiments.

As seen in FIG. 38, all analogs induce temporary activation of Erk½ within 10-20 min of treatment. However, despite some variability, it appears that 19C generates a slightly stronger and more prolonged response than other peptides. This may explain a higher proliferative potential of 19C observed in FIG. 37.

Analysis of the same samples for phosphorylation of another important signaling molecule implicated in cell proliferation and survival, Akt (PI3 kinase-Akt pathway), demonstrated an increase in Akt activation by all the analogs after 20 min of treatment which is sustained up to 3 h tested (FIG. 39).

In conclusion, all INGAP analogs demonstrate the ability to stimulate cell proliferation in RINm5 F cells, consistent with activation of Erk½ and Akt. 19C appears to be slightly more effective, as compared with the other analogs (both cell numbers and Erk½ data). One of the possible explanations is that 19C is more stable (up to 48 h in culture medium, vs 4-6 h half-life for 15 L. 15C and 19 L, based on the preliminary HPLC data). If this is the case, lowering of the 19C concentration would be an interesting extension of these experiments, especially in view of potential clinical applications.

Effect of INGAP analogs on cytokine-induced apoptosis in RIN-m5 F

Since Akt activation has been implicated in cell survival, we tested whether all INGAP analogs assert an anti-apoptotic effect, previously shown for the INGAP protein by a PhD student in our lab. Using the model of cytokine-induced apoptosis (IL1+INFg, both 10 nM), which is mediated by activation of NfkB pathway followed by an increase in iNOS production, we have tested the analogs in 5 independent experiments, at 167 nM concentration. As shown in FIG. 40, all analogs reduce the cytokine-induced iNOS expression roughly in half, which is overall similar to the INGAP protein, used as a positive control in this series of experiments.

Overall, the results of in vitro experiments show that each all INGAP analogs display a similar mode of action on HPDE and RINm5 F cells, with 19 being the most stable and potentially more effective analog than currently used 15 L. The data presented suggest that more studies are needed to investigate whether lower doses (and/or less frequent administration) of 19C makes it a more attractive islet regenerating drug.

Aim2:Biological Effects of INGAP Analog In Vivo, on the STZ-Induced Model of Diabetes in Mice.

In this part of the study, we used a well established model of STZ (streptozotocin)-induced diabetes, resulting in a substantial destruction of beta cell mass. Previous studies conducted in our lab have shown islet neogenic effects of INGAP-peptide (15 L) and INGAP protein after the 8-week administration daily.

Prior to the start of the STZ experiments, we tested the stability of INGAP analogs in the serum of non-diabetic mice by injecting 5-FAM labeled INGAP analogs (custom made at Canpeptide, Pointe Claire) at 500 mg/mouse IP . The analogs were FAM labeled to determine the post-injection time needed for the peptides to reach the pancreas and to potentially identify the target cells. Mice were sacrificed after 10 min, 30 min, 1 h and 2 h (three mice per group) and blood and tissue samples were collected for processing. Blood serum was precipitated with methanol (1:3) to remove serum proteins and the resulting supernatant was analyzed by HPLC to detect presence of INGAP peptides. FIG. 41 shows that all peptides disappear from the blood fairly rapidly, with both 15-mers undetectable after 30 min, and 19 L undetectable at 1 h. 19C was the longest lasting peptide of all, still detectable at 2 h, albeit at a very low level. These data confirm that 19C is the most stable analog.

Examination of formalin-fixed, paraffin-embedded tissue at 1 h, indicated that only FAM-19C was clearly detectable in the pancreas, whereas presence of other analogs was not certain due to a relatively high level of autofluorescence. These experiments need to be repeated by using cryosections vs paraffin section to minimize a potential loss of the fluorescence.

Referring to FIG. 42, C57BL/6 J mice (Charles River) 20-25 g, were rendered diabetic by a single injection of 150 mg/kg of STZ. The animals with blood glycemias of 15-30 mM after 7 days were used for the experiment. At 7 days, mice were randomized into 5 groups, 10 mice per group, equal representation of each glycemia level, (average glycemia per group being 24.4+0.0167 mM) and were treated with 500 mg of one of the analogs, IP, daily for 10 weeks. Blood glycemia values were taken weekly from the tip of the tail (fasting and/or non-fasting). The weight of animals was also taken weekly.

Of note, mice in all groups showed a high viability for 7 weeks, afterwards some losses of animals (13 mice) occurred but it was not always associated with hyperglycemia. The most likely other cause was inflammation associated with daily injections. This fact may have affected the average values of glycemia in most groups, except for 15C (no losses) and 19C (1 mouse lost). Decreasing frequency of injections would be therefore an important objective in the future experiments.

The weight of all diabetic mice proved to stay stable throughout, whereas Control Nondiabetic mice increased their weight by approximately 6 grams (data not shown).

As shown in FIG. 43, the average fasting glycemia gradually decreased in all groups. However, in this experiment, we did not observe a dramatic drop in glycemia (about 50%) seen in the previous study. The reasons for that are not well understood but it would be important to repeat this experiment on a different strain of mice or on mice procured from a different supplier.

It should be noted that in the 19C group, the decline in fasting glycemia appeared to occur earlier than in other groups, at 3 weeks of treatment.

After 10 weeks of treatment, the animals were sacrificed and the pancreata were weighed and processed for RNA extraction (still ongoing) and Immunocytochemistry.

As shown in FIG. 43, the average pancreatic weight of INGAP-treated mice was improved as compared to the control group. This was especially noticeable in 19C group, being equal to the weight of non-diabetic pancreata.

Pancreata harvested at the end of the treatments were fixed in formalin and paraffin sections were stained for hematoxylin and eosin (H&E) to evaluate the morphologic changes that would suggest islet neogenesis.

The analysis of the H&E stained sections showed that for all 4 analogues, and as previously described for INGAP-P (15 L), the pancreases show neogenesis features, i.e. the presence of islets bulging from or attached to ducts (FIG. 44) Of note, in 15C treated animals only 60% of the animals had these neogenesis features, the other 40% did not show any islets on the sections studied. In the other groups, the vast majority of animals display neoislets, versus virtually none in the PBS group.

Referring to FIG. 44, Histologic characteristics of the pancreas of streptozotocin-treated C57BL/6 J mice with and without INGAP analogs peptide treatment. H & E stained sections. A: The pancreas of a normal age-matched non-treated control mouse showing the histologic appearance of a normal islet for comparison. B,C: The pancreas of a saline-treated animal showing the absence of islets (most common feature (B), or the presence of a rare islet (1 per section, C). D: Pancreas of an INGAP-15 L peptide- treated animal showing an area of islet cell neogenesis, observed as endocrine-like cells budding from an adjacent ductule, normal-appearing islets. E: Pancreas of an INGAP-15C peptide-treated animal showing an area of islet cell neogenesis, observed as an islet budding from an adjacent duct. F: Some animals treated with INGAP-15C peptide did not show any islets, as in the PBS group. G: Pancreas of an INGAP-19 L peptide-treated animal showing an area of islet cell neogenesis, observed as an islet budding from an adjacent ductule. H: Pancreas of an INGAP-19C peptide-treated animal showing islet cell neogenesis, observed as endocrine-like cells budding from an adjacent ductule.

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosures as come within known or customary practice within the art to which the disclosure pertains and as may be applied to the essential features herein before set forth, and as follows in the scope of the appended claims.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

The contents of all documents and references cited herein are hereby incorporated by reference in their entirety.

What is claimed is:

1. A peptide comprising the sequence set forth in SEQ ID NO:4, SEQ ID NO. 5, or SEQ ID NO:6.

2. The peptide of claim 1, wherein the peptide induces pancreatic β-cell neogenesis, induces pancreatic β-cell regeneration, improves glucose homeostasis and/or reverses hyperglycemia in a subject.

3. An analog, homolog, fragment or variant of the peptide of set forth in claim 1, wherein the analog, homolog, fragment or variant has a biological activity of the peptide.

4. The analog, homolog, fragment or variant of claim 3, wherein the analog, homolog, fragment or variant has at least 80%, at least 85%, at least 90%, at least 95% at least 98%, or at least 99% sequence identity to the peptide.

5. The analog, homolog, fragment or variant of the fusion protein of claim 4, the biological activity is cell or receptor binding specificity of the peptide.

6. The analog, homolog, fragment or variant of the fusion protein of claim 4, wherein the biological activity is ability to induce pancreatic β-cell neogenesis, ability to induce islet cell regeneration, ability to improve glucose homeostasis and/or ability to reverse hyperglycemia in a subject.

7. A nucleic acid molecule comprising a nucleic acid sequence encoding the peptide or analog, homolog, fragment or variant thereof of SEQ ID NO:4, SEQ ID NO. 5, or SEQ ID NO:6.

8. The nucleic acid molecule of claim 7 operably linked to an expression control sequence to form an expression vector, wherein said expression vector is propagated in a suitable cell.

9. A pharmaceutical composition comprising the peptide or analog, homolog, fragment or variant of claim 1 and a pharmaceutically acceptable carrier or excipient.

10. The pharmaceutical composition of claim 9, wherein the composition is adapted for administration orally.

11. The pharmaceutical composition of claim 9, wherein the composition is adapted for administration by injection.

12. A method for preventing or treating a pancreatic condition or disease comprising administering the peptide or analog, homolog, fragment or variant thereof of the composition claim 9.

13. The method of claim 12, wherein the condition or disease is a metabolic disorder.

14. The method of claim 13, wherein the condition or disease is a β-cell associated disorder.

15. The method of claim 14, wherein the condition or disease is Type 1 diabetes, Type 2 diabetes or a complication of diabetes.

16. The method of claim 14 wherein β-cell death by apoptosis or necrosis is prevented or inhibited in the subject.

17. The method of claim 12, wherein the functionality of pancreatic cells is improved or restored in the subject.

18. The method of any one of claim 12, wherein plasma insulin levels are increased in the subject.

19. The method of any one of claim 14, wherein the number or size of pancreatic β-cells is increased in the subject, and/or wherein β-cell regeneration from pancreatic ductal cells is stimulated.

20. The method of any one of claim 14, wherein glucose homeostasis is restored or improved and/or hyperglycemia is reversed in the subject.

21. The method of claim 12 wherein said peptide or analog, homolog, fragment or variant thereof is administered by injection, orally, intravenously, intraperitoneally, intramuscularly or subcutaneously.

22. The method of claim 21, wherein said peptide or analog, homolog, fragment or variant thereof is administered orally, once-a-day.

23. The method of any one of claim 9, wherein the subject is a human.

24. The method of any one claim 9, wherein said peptide or analog, homolog, fragment or variant thereof is administered with a second therapeutic agent.

25. The method of claim 24, wherein the second therapeutic agent is administered concomitantly with said peptide or analog, homolog, fragment or variant thereof.

26. The method of claim 24, wherein said second therapeutic agent and said peptide or analog, homolog, fragment or variant thereof are administered sequentially.

27. The method of any one of claim 24, wherein said second therapeutic agent is a therapeutic for Type 1 or Type 2 diabetes.

28. The method of any one of claim 24, wherein said second therapeutic agent is anakinra.

29. A pharmaceutical composition for treatment of pancreatic insufficiency, comprising the peptide or analog, homolog, fragment or variant thereof of claim 1 and a pharmaceutically acceptable carrier or excipient.

30. The pharmaceutical composition of claim 29, wherein the peptide or analog, homolog, fragment or variant thereof is capable of stimulating β-cell regeneration from pancreatic ductal cells.

31. The pharmaceutical composition of claim 29 wherein the peptide or analog, homolog, fragment or variant thereof has a biological activity of mammalian INGAP protein.

32. The pharmaceutical composition of claim 31, wherein said biological activity is the ability to stimulate pancreatic duct-like cells or duct-associated cells to grow and proliferate.
